(19) United States
(12) Patent Application Publication
(10) Pub. No.: US 2018/0273972 A1
(43) Pub. Date: Sep. 27, 2018

(54) METHODS OF INCREASING VIRUS RESISTANCE IN CUCUMBER USING GENOME EDITING AND PLANTS GENERATED THEREBY

(71) Applicant: The State of Israel, Ministry of Agriculture & Rural Development, Argricultural Research Organiza, Rishon-LeZion (IL)

(72) Inventors: Amit GAL-ON, Herzlia (IL); Dalia WOLF, Nes Ziona (IL); Diana LIEBMAN, Ashdod (IL)

(21) Appl. No.: 15/781,509

(22) PCT Filed: Dec. 6, 2016
(86) PCT No.: PCT/IL2016/051309

§ 371 Date: Jun. 5, 2018

Related U.S. Application Data
(60) Provisional application No. 62/263,679, filed on Dec. 6, 2015.

(51) Int. Cl. C12N 15/82; A01H 5/08; A01H 6/34; C12N 15/11; C12N 9/22

(52) U.S. Cl. CPC C12N 15/8283; A01H 5/08; A01H 6/346; C12N 2800/80; C12N 9/22; C12N 15/8213; C12N 2310/20; C12N 15/11

(57) ABSTRACT

A cucumber plant comprising a genome being homozygous for a loss of function mutation in an eIF4E gene is provided. Also provided are methods of producing such plants.

### FIELD AND BACKGROUND OF THE INVENTION

The present invention, in some embodiments thereof, relates to methods of increasing virus resistance in cucumber using genome editing and plants generated thereby.

Plant viruses are known to cause extensive reductions in crop yields worldwide. Plant RNA viruses require host factors to maintain their life cycle. Many genes conferring resistance to viruses are recessive (Kang et al., 2005; Truniger and Aranda, 2009), including the eukaryotic translation initiation factors eIF4E or eIF(iso)4E (Lellis et al., 2002; Nicaise et al., 2003; Ruffel et al., 2006). The eIF4F-complex (eIF4E and eIF4G [or their isoforms] and eIF4A) and other host factors, such as the polyA-binding protein (PABP), bind to the potyviral 5′ m7G cap structure and 3′ polyA tail of mRNA for translation. In the eIF4F complex, the eIF4E and eIF(iso)4E genes link to the 5′ of mRNA or viral RNA and to the scaffold gene of each. Both, the eIF4E and eIF(iso)4E genes exist in plant cytoplasm and have redundant functions (Jackson et al., 2010; Sanfacçon, 2015; Wang and Krishnaswamy, 2012). Viruses, especially potyviruses, can associate with one or both of those proteins, through the viral-encoded protein VPg (Duprat et al., 2002; Hwang et al., 2009; Ling et al., 2009; Ruffel et al., 2006; Sato et al., 2005). The copy numbers of the eIF4E and eIF(iso)4E genes differ among plant species (Le Gall et al., 2011). In Cucumis spp. (cucumber and melon), one gene each of eIF4E and eIF(iso)4E have been identified (Rodríguez-Hernández et al., 2012; Gal-On et al., unpublished). Both eIF4E and eIF(iso)4E are recessive when mutated, and are essential for the translation of uncapped viruses having the VPg protein covalently linked to the viral RNA 5′ (Wittmann et al., 1997). Genes eIF4E and eIF(iso)4E interact with VPg in different hosts (Léonard et al., 2000; Sanfaçon, 2015; Jiang and Laliberte, 2011) and disruption of this link by mutagenesis or silencing prevents virus infectivity (Duprat et al., 2002; Lellis et al., 2002; Rodríguez-Hernández et al., 2012; Sato et al., 2005). The association of natural mutations in the eIF4E and eIF(iso)4E genes with potyvirus resistance has been observed in various crops and applied to breeding (Gómez et al., 2009). Broad RNA virus resistance has been demonstrated by silencing of the eIF4E gene in tomato and melon (Mazier et al., 2011; Rodríguez-Hernández et al., 2012).

Potyviridae cause significant losses in a wide range of crops. The viruses in this family have an RNA genome of approximately 10 kb with a 3′ poly A tail that encodes a polyprotein, which is cleaved by three viral proteases resulting in 9-11 putative mature proteins (Revers and García, 2015). The VPg is the amino part of the NIa protease, which is covalently attached to the genomic RNA 5′ end as an mRNA cap analogue. VPg plays a role in polyprotein translation and other function in the virus-life cycle. Mutations in the VPg gene have been shown to be associated with breaking natural virus resistance by number of viruses (Ayme et al., 2006; Hébrard et al., 2006; Moury et al., 2004). Other potyvirus genes such as cylindrical inclusion and P1 are known to be involved with breaking eIF4E-mediated resistance (Abdul-Razzak et al., 2009; Nakahara et al., 2010).

### SUMMARY OF THE INVENTION

According to an aspect of some embodiments of the present invention there is provided a cucumber plant comprising a genome being homozygous for a loss of function mutation in an eIF4E gene.

According to an aspect of some embodiments of the present invention there is provided a method of increasing viral resistance in a cucumber plant, the method comprising subjecting a genome of the cucumber plant to a DNA editing agent so as to induce a loss of function mutation in an eIF4E gene so as to impart recessive resistance resultant of the loss of function mutation.

According to some embodiments of the invention, the method further comprises subjecting the cucumber plant comprising the loss of function in the eIF4E gene to at least one step of crossing or selfing.

According to some embodiments of the invention, the plant comprises a heterologous nucleic acid sequence encoding an endonuclease of a genome editing agent.

According to some embodiments of the invention, the cucumber plant exhibiting higher resistance to an RNA virus as compared to that of a wild-type cucumber plant of the same genetic background.

According to some embodiments of the invention, the RNA virus comprises a plurality of viruses comprising CVYV, ZYMV and PRSV-W.

According to some embodiments of the invention, the RNA virus is an uncapped virus having the VPg protein covalently linked to the viral RNA 5′.

According to some embodiments of the invention, the virus belongs to the potyviridae family.

According to some embodiments of the invention, the virus is selected from the group consisting of CVYV, ZYMV and PRSV-W.

According to some embodiments of the invention, the resistance is manifested by absence, delayed or milder symptoms appearance.

According to some embodiments of the invention, the resistance is manifested by no or reduced accumulation of RNA of the virus or by visual monitoring of symptoms.

According to some embodiments of the invention, the DNA editing agent is directed to exon 1 of a coding sequence of the eIF4E.

According to some embodiments of the invention, the DNA editing agent is directed to exon 3 of a coding sequence of the eIF4E.

According to some embodiments of the invention, the DNA editing agent does not edit the coding sequence of eIF(iso)4E.

According to some embodiments of the invention, the loss of function mutation is selected from the group consisting of a deletion, an insertion, and insertion/deletion (indel) and a substitution.

According to an aspect of some embodiments of the present invention there is provided a plant part comprising DNA of the plant of any one of claims 1 and 4-15.

According to some embodiments of the invention, the plant part is a seed or a fruit.

According to an aspect of some embodiments of the present invention there is provided a nucleic acid construct comprising a nucleic acid sequence coding for a DNA editing agent capable of hybridizing to an eIF4E gene of a cucumber and facilitating editing of the eIF4E gene, the nucleic acid sequence being operably linked to a cis-acting regulatory element for expressing the DNA editing agent in a cell of a cucumber.

According to some embodiments of the invention, the nucleic acid construct further comprises a nucleic acid sequence encoding an endonuclease.

According to some embodiments of the invention, the method comprises breeding out the DNA editing agent.

According to some embodiments of the invention, the method comprises selecting a cucumber plant wherein the DNA editing agent and the eIF4E gene are on different chromosomes prior to the breeding out.

According to some embodiments of the invention, the DNA editing agent is of a DNA editing system selected from the group consisting of meganucleases, Zinc finger nucleases (ZFNs), transcription-activator like effector nucleases (TALENs) and CRISPR.

According to some embodiments of the invention, the DNA editing agent is of CRISPR/Cas-9.

According to some embodiments of the invention, the DNA editing agent comprises a guide RNA (sgRNA) selected from the group consisting of SEQ ID NOs: 51-254 or 255-256.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of the map of the binary vector with Cas9-sgRNA.

FIGS. 2A-C show gene editing of eIF4E mediated by CRISPR/Cas9 in transgenic cucumber plants (SEQ ID NOs: 257-260). (FIG. 2A) Schematic representation of the cucumber eIF4E genomic map and the sgRNA1 and sgRNA2 target sites (red arrows). The target sequence is shown in red letters together with the restriction site (underlined), and the PAM motif is marked in bold underlined letters. The black arrows indicate the primers flanking the target sites used to detect the mutations. (FIG. 2B) Restriction analysis of T0 PCR fragments of CEC-1, CEC1-4 and CEC2-5. (FIG. 2C) Alignment of 9 colony sequences from the undigested fragment of line 1 with the wild-type (wt) genome sequence. DNA deletions are shown in red dashes and deletion sizes (nt) are marked on the right side of the sequence.

FIGS. 3A-B show the genotyping of eIF4E mutants in representative T1 progeny plants of CEC1-1 (SEQ ID NOs: 261-269). (FIG. 3A) PCR restriction analysis of Cas9/sgRNA1-mediated mutations (upper-panel) and transgene insertion (lower panel) in 10 representative T1 cucumber plants and non-mutant wild-type (wt). (FIG. 3B) Alignment of 4 representative eIF4E mutant plants with wild-type sequence. Sequences of each plant represented clones from undigested fragments. The target sequence is shown in red letters and the PAM motif marked by bold underlined letters. DNA deletions are marked in red dashes and deletion sizes (nt) are indicated on the right side of the sequence.

FIGS. 4A-B show the genotyping of eIF4E mutants in representative T1 progeny plants of line CEC1-4 (SEQ ID NOs: 270-288). (FIG. 4A) PCR restriction analysis of Cas9/sgRNA1-mediated mutations (upper-panel) and transgene insertion (lower panel) in 8 T1 cucumber plants and non-mutant plant wild type (wt). (FIG. 4B) Alignment of 3 eIF4E transgenic mutant plants 4, 5, and 6 with wild-type sequence. Sequences of each plant represent clones from undigested fragments. The target sequence is shown in red letters and the PAM motif marked in bold underlined letters. DNA deletions are marked by red dashes and deletion sizes (nt) are indicated on the right side of each sequence.

FIGS. 5A-B show the genotyping of the Cas9/sgRNA2-mediated mutation in T1 progeny plants of CEC2-5 line (SEQ ID NOs: 289-300). (FIG. 5A) PCR restriction analysis of Cas9/sgRNA2-mediated mutations (upper-panel) and the presence of the Cas9/sgRNA2 transgene (lower panel) in 8 representative T1 cucumber plants. (FIG. 5B) Alignment of four representative eIF4E mutants plants with the wild-type sequence. The target sequence is shown in red letters, the PAM motif marked in bold underlined letters. DNA deletions or insertions are marked by red dashes and letters and the size of deletions or insertions (nts) are indicated on the right side of the sequence.

FIGS. 6A-B show that homozygous eif4e mutant plants exhibit immunity to CVYV infection. (a) Disease symptoms (leaves and plants) of heterozygous (Het-mut), homozygous (Hom-mut) and non-inoculated plants (control) of CEC1-1-7-1 T3 generation, 10 dpi. (b) RT-PCR analysis of CVYV RNA accumulation 14 dpi in homozygous eIF4E mutant plants (plants 1 to 11), heterozygous eIF4E mutant plant (Het.) and non-inoculated plants (control) 14 dpi. The TIP41 (tonoplast intrinsic protein) was used as a reference gene for RT-PCR amplification. A molecular marker 100-bp ladder is shown (M).

FIGS. 7A-C show that homozygous eif4e mutant plants exhibit resistance to ZYMV infection. (FIG. 7A) Disease symptoms of heterozygous (Het-mut), homozygous (Hom-mut) and non-inoculated plants (control) of the CEC1-1-7-1 T3 generation 25 dpi. (FIG. 7B) RT-PCR analysis of ZYMV RNA accumulation in homozygous eIF4E mutant plants (1 to 10), heterozygous plants (Het-mut) and non-inoculated plants (H) at 14 dpi. Tip41 was used as a reference gene for RT-PCR amplification. Molecular marker of 100-bp ladder (M). (FIG. 7C) Relative (qRT-PCR) ZYMV RNA accumulation in CEC1-1-7-1 heterozygous (Het-mut) and 2 classes of homozygous mutants: resistant (resistant) and breaking (Break). RNA was extracted from three plants (third top leaf) and the ZYMV level was calculated using the ΔΔCt method normalized to F-box gene expression level.

FIGS. 8A-C show that homozygous eif4e mutants exhibit resistance to PRSV-W infection. (FIG. 8A) Disease symptoms of heterozygous (Het-mut), homozygous (Hom-mut) and non-inoculated (control) plants of CEC1-1-7-1 T3 generation 21 dpi. (FIG. 8B) RT-PCR analysis of PRSV-W RNA accumulation in homozygous plant (plants 1 to 8), heterozygous plant (Het.) and non-inoculated plants (H) 14 dpi. Tip41 was used as a reference gene for RT-PCR amplification. Molecular marker 100-bp ladder (M). (FIG. 8C) Relative (qRT-PCR) accumulation of PRSV-W RNA in CEC2-5-M-9 heterozygous (Het-mut), and 3 classes of homozygous mutants: resistant (resistant), breaking (Break) and recovering (Recovery). RNA was extracted from the second top leaf of 3 plants and the PRSV-W RNA level was calculated using the ΔΔCt method normalized to F-box gene expression level.

### DESCRIPTION OF SPECIFIC EMBODIMENTS OF THE INVENTION

The present invention, in some embodiments thereof, relates to methods of increasing virus resistance in cucumber using genome editing and plants generated thereby.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Whilst conceiving embodiments described herein, and reducing them to practice, the present inventor developed virus resistance in cucumber (Cucumis sativus L.) by utilizing Cas9/sgRNA technology to disrupt the recessive eIF4E gene function. Cas9/sgRNA constructs were targeted to the N′ and C′ terminus of the coding sequence of the eIF4E gene. Small deletions and SNPs were observed in the eIF4E gene targeted sites of T1 generation transformed cucumber plants, but not in putative off-target sites. Non-transgenic heterozygous eIF4E mutant plants were selected for production of non-transgenic homozygous T3 generation plants. Homozygous T3 progeny following Cas9/sgRNA that had been targeted to both eIF4E sites exhibited immunity to Cucumber vein yellowing virus (ipomovirus) infection and resistance to the potyviruses Zucchini yellow mosaic virus and Papaya ring spot mosaic virus-W. In contrast, heterozygous-mutant and non-mutant plants were highly susceptible. For the first time, virus resistance has been developed in the cucumber crop, non-transgenically, not visibly affecting plant development, and without long-term backcrossing.

Thus, according to an aspect of the invention there is provided a method of modifying a genome of a cucumber cell or plant, the method comprising subjecting a genome of the cucumber cell or plant to a DNA editing agent so as to induce a loss of function mutation in at least one allele of a eIF4E gene in the genome of the cucumber.

As used herein a “cucumber” refers to material that is essentially of species Cucumis sativus.

As used herein “plant” refers to whole plants, a grafted plant, ancestors and progeny of the plants and plant parts, including seeds, fruits, shoots, stems, roots (including tubers), rootstock, scion, and plant cells, tissues and organs.

According to a specific embodiment, the cell is a germ cell.

According to a specific embodiment, the cell is a somatic cell.

The plant may be in any form including suspension cultures, embryos, meristematic regions, callus tissue, leaves, gametophytes, sporophytes, pollen, and micro spores.

According to a specific embodiment, the plant part comprises DNA.

According to a specific embodiment, the cucumber plant is of a diploid cucumber breeding line, more preferably an elite line.

According to a specific embodiment, the cucumber plant is of an elite line.

According to a specific embodiment, the cucumber plant is of a purebred line.

According to a specific embodiment, the cucumber plant is of a cucumber variety or breeding germplasm.

The term “breeding line”, as used herein, refers to a line of a cultivated cucumber having commercially valuable or agronomically desirable characteristics, as opposed to wild varieties or landraces. The term includes reference to an elite breeding line or elite line, which represents an essentially homozygous, usually inbred, line of plants used to produce commercial F₁ hybrids. An elite breeding line is obtained by breeding and selection for superior agronomic performance comprising a multitude of agronomically desirable traits. An elite plant is any plant from an elite line. Superior agronomic performance refers to a desired combination of agronomically desirable traits as defined herein, wherein it is desirable that the majority, preferably all of the agronomically desirable traits are improved in the elite breeding line as compared to a non-elite breeding line. Elite breeding lines are essentially homozygous and are preferably inbred lines.

The term “elite line”, as used herein, refers to any line that has resulted from breeding and selection for superior agronomic performance. An elite line preferably is a line that has multiple, preferably at least 3, 4 5, 6 or more (genes for) desirable agronomic traits as defined herein.

The terms “cultivar” and “variety” are used interchangeable herein and denote a plant with has deliberately been developed by breeding, e.g., crossing and selection, for the purpose of being commercialized, e.g., used by farmers and growers, to produce agricultural products for own consumption or for commercialization (fresh consumption, processing, feed, etc). The term “breeding germplasm” denotes a plant having a biological status other than a “wild” status, which “wild” status indicates the original non-cultivated, or natural state of a plant or accession.

The term “breeding germplasm” includes, but is not limited to, semi-natural, semi-wild, weedy, traditional cultivar, landrace, breeding material, research material, breeder's line, synthetic population, hybrid, founder stock/base population, inbred line (parent of hybrid cultivar), segregating population, mutant/genetic stock, market class and advanced/improved cultivar. As used herein, the terms “purebred”, “pure inbred” or “inbred” are interchangeable and refer to a substantially homozygous plant or plant line obtained by repeated selfing and-or backcrossing.

A non-comprehensive list, of cucumber varieties is provided hereinbelow:

### Aardvark Cucumber

### Babylon

### Bella

### Burpless

### Burpless Tasty Green

### Bush Crop

### Camaro (European)

### Carmen

### Cobra

### Cool Breeze (India)

### Corinto

### Cortez

### Crystal Apple

### Cucino

### Cutter

### Darlington

### Dasher II

### Dayton

### Delizia

### Diamante

### Diomede

### Diva

### Dominator

### Exocet

### Fanfare

### Fountain

### Garden Sweet Burpless

### General Lee

### Genuine

### Gherkin

### Green Finger

### Green Slam

### Greensleeves

### H-19 Little Leaf

### Harmonie

### HMX 8416

### Impact

### Indio

### Indy

### Intimidator

Jawell (mini)

### Jogger

### Katrina

### Laura

### Lightening

### Lider

### Lucky Dance

### Marketmore 76

### Marketmore 97

### Masterpiece

### Munchmore

### Niagara

### Northern Pickling

### Olympian

### Orient Express

### Panther

### Picolino

### Poinsett

### Poinsett 76

### Pot Luck

### PS14710903

### Purples Tasty Green

### Raider

### Rockingham

### Rocky

### Saber

### Salad Bush

### Salt and Pepper

### Shantung Suhyo Cross

### Sikkim

### Slicemaster Select

### Soarer

### Socrates

### Spacemaster

### Speedway

### Sprint 440

### SR2389CW

### SRQ2387

### SRQ2389

### Stonewall

### Straight Eight Elite

### Sultan

### Summer Dance

### Summer Top

### Suyo Long

### Sweet Slice

### Sweet Success

### Sweeter Yet

### Swing

### Talladega

### Tasty Green

### Tasty Jade

### Taurus

### Telegraph

### Thunder

### Thunderbird

### Tiffany

### Triumph

### Turbo

### Tyria

### Vega

### Viper

### White Revolver

### Yaniv

### Zeina

### Zipangu

As used herein “modifying a genome” refers to introducing at least one mutation in at least one allele of an eIF4E gene of the cucumber. According to some embodiments, modifying refers to introducing at least two mutations in the two alleles of the eIF4E gene of the cucumber. According to at least some embodiments, the mutations on the two alleles of the eIF4E gene are in a homozygous form.

According to some embodiments, the mutations on the two alleles of the eIF4E gene are noncomplementary.

As used herein “eIF4E” refers to the gene encoding the translation initiation product eIF4E in cucumber, e.g., SEQ ID NO: 1, 8, eIF4E—accession no. XM_004147349, XM_004147349.1).

eIF4E does not refer to eIF(iso)4E (accession no. XM_004147116.2) (SEQ ID NO: 2) which is encoded by a different gene than eIF4E.

According to a specific embodiment, the loss of function mutation (homozygous or non-complementary) in an eIF4E gene of the cucumber does not comprise mutations in the gene encoding to eIF(iso)e4.

According to a specific embodiment, the DNA editing agent modifies the target sequence eIF4E and is devoid of “off target” activity, i.e., does not modify other sequences in the cucumber genome.

According to a specific embodiment, the DNA editing agent comprises an “off target activity” on a non-essential gene in the cucumber genome.

Non-essential refers to a gene that when modified which the DNA editing agent does not affect the phenotype of the target genome in an agriculturally valuable manner (e.g., biomass, vigor, yield, selection, biotic/abiotic stress tolerance and the like).

Off-target effects can be assayed using methods which are well known in the art and are described hereinbelow.

As used herein “loss of function” mutation refers to a genomic aberration which results in the inability of eIF4E to contribute to viral infection, via protein translation.

According to a specific embodiment, the loss of function mutation results in no expression of the eIF4E mRNA or protein.

According to a specific embodiment, the loss of function mutation results in expression of an eIF4E protein which is not capable of supporting (contributing to) viral infection.

According to a specific embodiment, the loss of function mutation is selected from the group consisting of a deletion, insertion, insertion-deletion (Indel), inversion, substitution and a combination of same (e.g., deletion and substitution e.g., deletions and SNPs).

According to a specific embodiment, the loss of function mutation is smaller than 10 Kb.

According to a specific embodiment, the “loss-of-function” mutation is in the 5′ of eIF4E to inhibit the production of any eIF4E expression product (e.g., exon 1).

According to a specific embodiment, the loss-of-function” mutation anywhere in the eIF4E that allows the production of an eIF4E expression product (e.g., exon 3), while being unable to facilitate (contribute to) viral infection. Also provided herein is a mutation in regulatory elements of the gene e.g., promoter.

Table 1 below provides suggested positions.

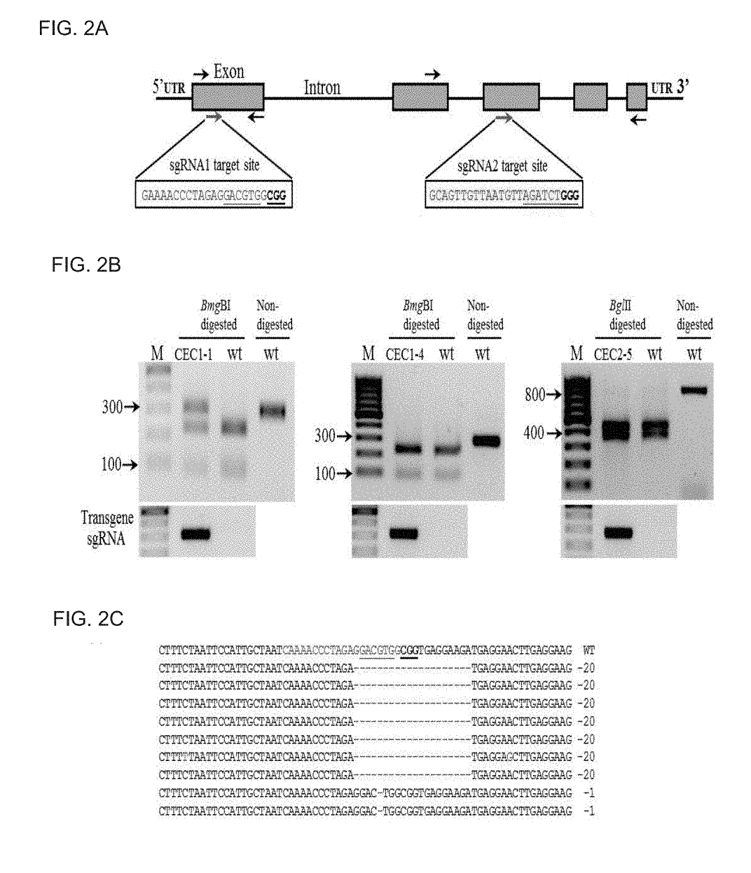

FIGS. 2-5 provides exemplary events for loss of function mutations that are contemplated herein.

As mentioned the cucumber plant comprises the loss of function mutation in at least one allele of the eIF4E gene.

Recessive resistance arising from the loss of a host factor necessitates mutations in both allele of the eIF4E gene.

According to a specific embodiment, the mutation is homozygous.

According to an aspect of the invention there is provided a method of increasing viral resistance in a cucumber plant, the method comprising subjecting a genome of the cucumber plant to a DNA editing agent so as to induce a loss of function in an eIF4E gene so as to impart recessive resistance resultant of said loss of function mutation.

As used herein “viral resistance” is to a virus that uses host eIF4E translation initiation factor for its infection (virus life cycle).

According to a specific embodiment, the virus is an RNA virus.

According to a specific embodiment, the virus is an RNA virus being an uncapped virus having the VPg viral encoded protein covalently linked to the viral RNA 5′.

According to a specific embodiment, the virus belongs to the potyviridae family.

According to a specific embodiment, the virus is an Ipomovirus.

According to a specific embodiment, the virus is selected from the group consisting of CVYV (Cucumber vein yellowing virus), ZYMV (Zucchini yellow mosaic virus), PRSV-W (Papaya ring spot mosaic virus-W) and WMV (Watermelon mosaic virus).

According to a specific embodiment, the virus is selected from the group consisting of CVYV and ZYMV.

According to a specific embodiment, the plant is resistant to a plurality of viruses e.g., CVYV and ZYMV, or PRSV-W, CVYV and ZYMV. It will be appreciated that when present only on one allele, the present teachings further contemplate either repeating the DNA editing step and/or subjecting the cucumber plant comprising said loss of function mutation in said one allele to at least one step of crossing or selfing so as to obtain a cucumber plant comprising said loss of function mutation in two alleles of said eIF4e gene and having increased resistance to viral infection.

As used herein “increased resistance” refers to at least 10%, 20%, 30%, 40%, 50%, 60%, 70%, 80%, 85%, 90% or even 95%, increase in viral resistance as compared to that of a cucumber plant of the same genetic background not comprising the loss of function mutation (or having it in a heterozygous form) and as manifested by either delayed or milder symptoms appearance or reduced accumulation of RNA of the virus, as assayed by methods which are well known in the art (see Examples section which follows).

According to a specific embodiment, increased resistance is evidenced for at least 30 days.

Following is a description of various non-limiting examples of methods and DNA editing agents used to introduce nucleic acid alterations to a gene of interest and agents for implementing same that can be used according to specific embodiments of the present disclosure.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDS) and non-homologous end-joining (NHEJF). NHEJF directly joins the DNA ends in a double-stranded break, while HDR utilizes a homologous donor sequence as a template for regenerating the missing DNA sequence at the break point. In order to introduce specific nucleotide modifications to the genomic DNA, a donor DNA repair template containing the desired sequence must be present during HDR.

Genome editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the non-homologous end-joining (NHEJ) pathway often results in small deletions or small sequence insertions. Since each repair made by NHEJ is unique, the use of a single nuclease pair can produce an allelic series with a range of different deletions at the target site.

The deletions typically range anywhere from a few base pairs to a few hundred base pairs in length, but larger deletions have been successfully generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

CRISPR-Cas system (also referred to herein as “CRISPR”)—Many bacteria and archea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.).

It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded breaks produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of a gRNA that can be used in the present disclosure include those described in the Example section which follows.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene. Use of clustered regularly interspaced short palindromic repeats (CRISPR)-associated (Cas)-guide RNA technology and a Cas endonuclease for modifying plant genomes are also at least disclosed by Svitashev et al., 2015, Plant Physiology, 169 (2): 931-945; Kumar and Jain, 2015, J Exp Bot 66: 47-57; and in U.S. Patent Application Publication No. 20150082478, which is specifically incorporated herein by reference in its entirety.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRY”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

According to a specific embodiment, the DNA editing agent is CRISPR-Cas9. Exemplary gRNA sequences are provided in Table 1 hereinbelow (named CECsgRNA1 and CECsgRNA2).

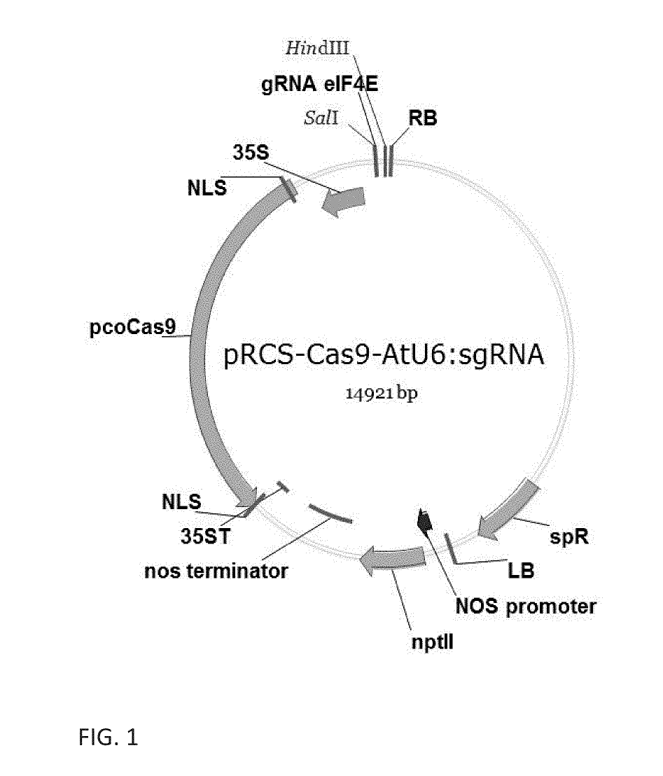

The DNA editing agent is typically introduced into the plant cell using expression vectors (e.g., binary vector), see for instance FIG. 1.

Thus, according to an aspect of the invention there is provided a nucleic acid construct comprising a nucleic acid sequence coding for a DNA editing agent capable of hybridizing to an eIF4E gene of a cucumber and facilitating editing of said eIF4E gene, said nucleic acid sequence being operably linked to a cis-acting regulatory element for expressing said DNA editing agent in a cell of a cucumber.

Embodiments of the invention relate to any DNA editing agent, such as described above.

According to a specific embodiment, the DNA editing agent is CRISPR/Cas9 sgRNA (or also as referred to herein as “gRNA”).

According to a specific embodiment, said nucleic acid construct further comprises a nucleic acid sequence encoding an endonuclease of a DNA editing agent (e.g., Cas9 or the endonucleases described above).

According to another specific embodiment, the endonuclease and the sgRNA are encoded from different constructs whereby each is operably linked to a cis-acting regulatory element active in plant cells (e.g., promoter).

In a particular embodiment of some embodiments of the invention the regulatory sequence is a plant-expressible promoter.

Constructs useful in the methods according to some embodiments may be constructed using recombinant DNA technology well known to persons skilled in the art. Such constructs may be commercially available, suitable for transforming into plants and suitable for expression of the gene of interest in the transformed cells.

As used herein the phrase “plant-expressible” refers to a promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ. Examples of promoters useful for the methods of some embodiments of the invention include, but are not limited to, Actin, CANV 35S, CaMV19S, GOS2. Promoters which are active in various tissues, or developmental stages can also be used.

Nucleic acid sequences of the polypeptides of some embodiments of the invention may be optimized for plant expression. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest (in this case cucumber). Therefore, an optimized gene or nucleic acid sequence (e.g., encoding Cas9) refers to a gene in which the nucleotide sequence of a native or naturally occurring gene has been modified in order to utilize statistically-preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1 N [(Xn−Yn)/Yn]2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants is compiled using the data of Murray et al. (1989, Nuc Acids Res. 17:477-498).

Plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced. Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

It will be appreciated that following transformation, the transgene encoding the transgene (i.e., DNA editing agent e.g., Cas9) is typically breeded our or selected out such that the resultant crossing/selfing comprises the loss of function mutation(s) in the two alleles of the eIF4E and no DNA editing agent. Crossing/selfing may be repeated as needed.

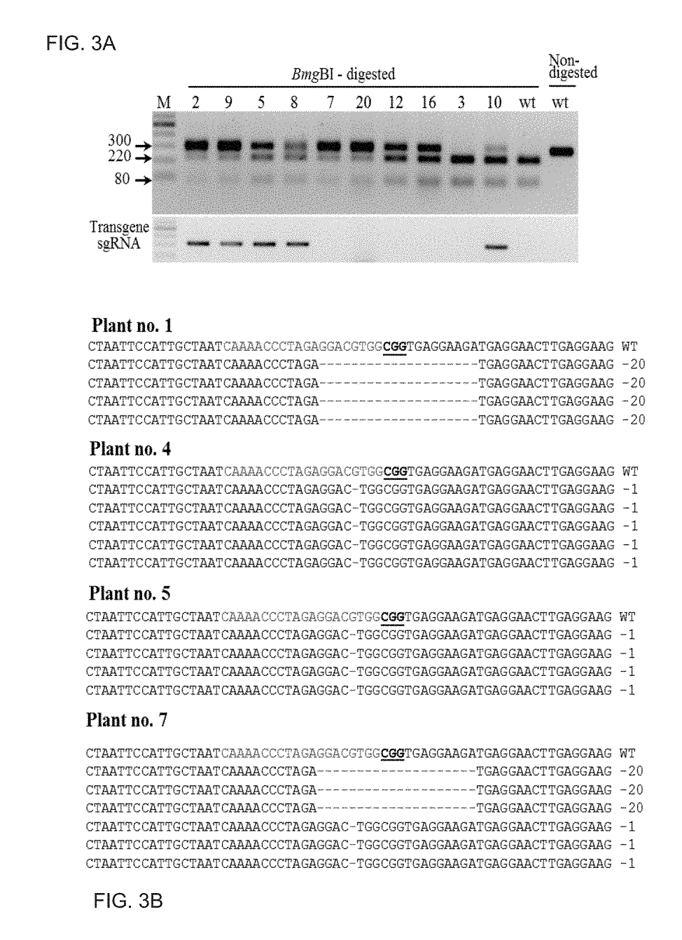

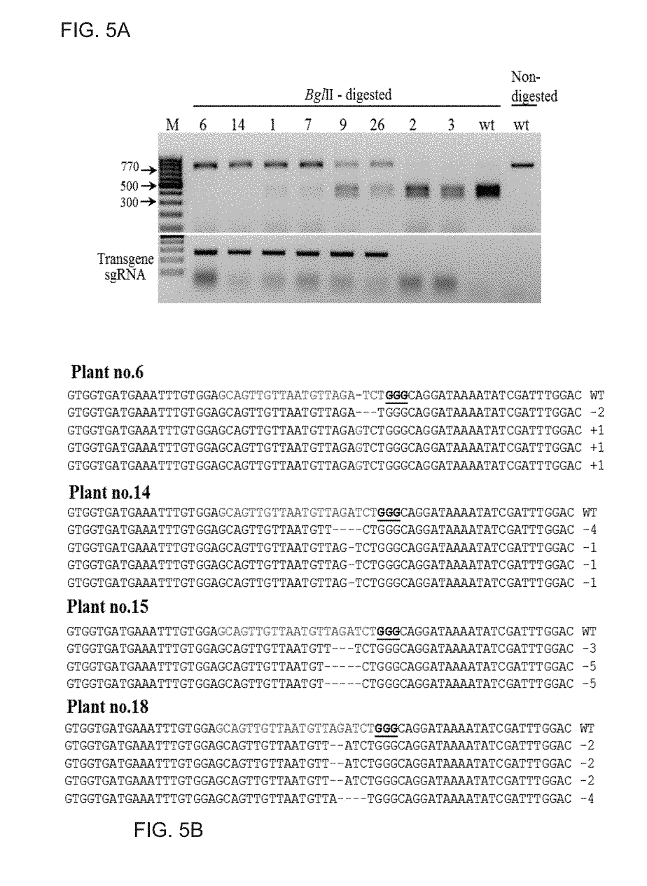

Thus, according to a specific embodiment, plants selected following transformation with the DNA editing agent (e.g., CRISPR/Cas9) are those exhibiting independent segregation of the DNA editing agent and the mutated eIF4E at F₁ or F₂ for instance plant 7 and 4 (FIGS. 3A-B) and plant 6 and 14 (FIG. 5A-B).

Thus, according to a specific embodiment, the DNA editing agent and the eIF4E gene are on different chromosomes prior to the breeding out.

Cucumbers generated according to the present teachings may find many uses in various industries including the food, cosmetics (e.g., masks, scrubs, shampoo, creams, gels, ointments), fragrance, antiseptic and chemical industries.

Thus, the present teachings also relate to parts of the plants as described herein or processed products thereof.

According to a specific embodiment, such processed products comprise DNA including the mutated eIF4E gene that imparts the recessive resistance.

It is expected that during the life of a patent maturing from this application many relevant DNA editing agents will be developed and the scope of the term DNA editing agent is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

For example, a given SEQ ID NO: is expressed in a DNA sequence format (e.g., reciting T for thymine), but it can refer to either a DNA sequence that corresponds to a given nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Experimental Procedures

CRISPR/Cas9 Binary Construct Design

The pRCS binary vector (Dafny-Yelin and Tzfira, 2007) was used which comprised 35S:Cas9-AtU6:sgRNA-PDS, where the Cas9 gene was optimized for Arabidopsis plant codon usage (Li et al., 2013; Nekrasov et al., 2013). The nptII (kanamycin) selection marker gene under the control of the 35S promoter and nos terminator was cloned into the AscI site (FIG. 1).

eIF4E sgRNA Design and Cloning

The eIF4E gene (GenBank accession no. XM_004147349, SEQ ID NO: 1) of cucumber (Cucumis sativus) was selected as the target gene. Two different sgRNA forward primers were designed for the eIF4E target gene (Table 1).

TABLE 1
List of Primers and their Application

| | Sequence (target sequences are shown | |
|---|---|---|
| | in bold and restriction sites are | |
| Primer name | underlined)/SEQ ID NO: 3-45 | Purpose |
| Cuc-EFsgRNAl-SalI | agagtcgacatagcgattgaaaaccctagaggacgtgg | Construction of pRCS-35S: |
| Fwd (BmgBI) | gttttagagctagaaatagca | Cas9-AtU6:CECsgRNA1 |
| CECsgRNA1 | Gaaaaccctagaggacgtgg | eIF4E target sequence |
| Cuc-EFsgRNA2-SalI | agagtcgacatagcgattgcagttgttaatgttagatctgtt | Construction of pRCS-35S: |
| Fwd (BglII) | ttagagctagaaatagca | Cas9-AtU6:CECsgRNA2 |
| CECsgRNA2 | gcagttgttaatgttagatct | eIF4E trget sequence |
| U6-sgRNA HindIII Rev | gctaagctt agaactaaaaaaagcac | Construction of pRCS-35S: |
| | | Cas9-AtU6:CECsgRNA1/sgRNA2 |
| Gene & Accession No. | Primer name and Sequence | Purpose |
| elF4E Cucumber | Fwd gctctagaatggtagttgaagatacgatc | Cloning of elF4E gene |
| XM_004147349.1 | Rev gcgcggccgctcacaccatatatttattc | |
| Cas9 | Fwd gcgtatccgttcttagactg | To verify transgene |
| | Rev ggttgtaggtctgaacaagc | |
| sgRNA | Fwd gacactgacggctttatgcc | To verify transgene |
| | sgRNA1 Rev gtgctttttttagatctaagcttagc | |
| Cuc-elF4E (sgRNA1) | sgEF1 Fwd atggtagttgaagatacgatc | PCR/Restriction analysis for |
| | sgEF1 Rev ctccagaactcctcgacagt | mutation detection; sequencing |
| Cuc-elF4E (sgRNA2) | sgRNA2 Fwd cgttgagggcagatttgtac | PCR/Restriction analysis for |
| | sgRNA2 Rev tattcttcgcatgtctatca | mutation detection; sequencing |
| Cuc-elF4E | Fwd caaaaccctagatgaggaact | Screening for mutations of 20 |
| | Rev ctccagaactcctcgacagt | nt deletions in sgRNA1 mutant |
| | | plants |
| CVYV Coat protein | Fwd attcccaagctcagcaaaga | RT-PCR |
| (CP) AY290865 | Rev cgaacctttctatctcccaatg | |
| ZYMV HC-Pro | Fwd acatgcatctgactggtgag | RT-PCR |
| EF062582 | Rev agttgcaacatccatcaatgaa | |
| ZYMV NIb EF062582 | Fwd agctccatacatagctgagaca | qRT-PCR analysis |
| | Rev gaaccaagaggcgaattgct | |
| PRSV-W Israeli CP | Fwd gaatggtacatcaccggacata | qRT-PCR analysis |
| | Rev cggagtggcatgctctatta | |
| CMV-Fny CP D10538 | Fwd ctgatctgggcgacaagga | RT-PCR |
| | Rev cgataacgacagcaaaacac | |
| CGMMV CP KF155232 | Fwd gtttcgcttctcagctccac | RT-PCR |
| | Rev cgcgtcatcagtacgcttta | |
| F-box-cucumber | Fwd ggttcatctggtggtctt | Reference gene for |
| csa881870 | Rev ctttaaacgaacggtcagtcc | qRT-PCR analysis |
| TIP 41-cucumber | Fwd caacaggtgatattggattatgattatac | Reference gene for |
| GW881871 | Rev gccagctcatcctcatataag | qRT-PCR analysis |
| scaffo1d02925:+101443 | Fwd gaacgccctggaaaacggac | Off-target analysis |
| | Rev ggccattggtgttttcaaac | |
| scaffold00919:1885568 | Fwd agaaggactacattattagagag | Off-target analysis |
| | Rev tttaaagtaagatcttgaatgatc | |
| scaffo1d00995:4267 | Fwd caaccaccaccaaccgacat | Off-target analysis |
| | Rev ggatgttgagatcatcttga | |
| scaffold02925:041731 | Fwd atgacatgcccatctcgcct | Off-target analysis |
| | Rev cacaaggaagattaagtgaga | |
| scaffold03356:+3916928 | Fwd aaagttacaaatgttggaagaca | Off-target analysis |
| | Rev aatgagttgctactagaccc | |

Each primer contained a SalI site as part of the U6 Arabidopsis promoter (FIG. 1). The eIF4E target sequence along with the sgRNA scaffold was amplified using sgRNA1 or sgRNA2 as a forward primer (Table 1) and a reverse primer of the PolIII-terminator sequence that contained a HindIII site and pRCS-35S:Cas9-AtU6:sgRNA-PDS as a template. The amplified DNAs (˜80 bp) were cloned into SalI and HindIII sites of the pRCS-35S:Cas9-AtU6:sgRNA-nptII binary plasmid (Figure S1). The obtained clones were confirmed by sequencing.

Agrobacterium-Mediated Transformation

Agrobacterium tumefaciens-mediated transformation of cucumber ‘Ilan’ (Zera'im Gedera, Israel) was performed according to (Gal-On et al., 2005). Cut cotyledons without embryo were pre-cultured for one day followed by inoculation with A. tumefaciens EHA105 containing the CRISPR/cas9 constructs (pRCS-35S:Cas9-AtU6:CECsgRNA1 or CECsgRNA2). The cotyledon segments were transferred to a selective regeneration medium that contained 100 mg/l kanamycin. Shoots regenerated from explants were transferred to an elongation medium, followed by a rooting medium with 100 mg/l kanamycin. Well-rooted plants were transferred to moist Jiffy 7 peat pellets and covered with transparent plastic boxes for hardening in a growth chamber under continuous white fluorescent light at 25° C.

Transgenic Plant Growth Conditions and Propagation.

Transgenic lines were transferred to coir medium (Pelemix Ltd., USA) 2-3 weeks post hardening and grown in greenhouse conditions under natural daylight at 26° C. Water and fertilizer (120 ppm of 5:3:8 NPK) were supplied twice daily by drip irrigation according to the size of the plants. T0 transgenic cucumber lines were hand-pollinated with male flowers of the monoecious ‘Bet Alfa’, because the transformed ‘Ilan’ is gynoecious.

Genotyping and Mutant Verification

Genomic DNA was isolated from T0 transgenic and non-transgenic cucumber plants by Gen Elute™ Plant Genomic DNA Miniprep kit (Sigma Aldrich) and by Dellaporta method (Dellaporta et al., 1983). The presence of the Cas9/sgRNA1/sgRNA2 transgene in T0 lines was confirmed by PCR using specific primers (Table 2).

TABLE 2
Mutations in the putative eIF4E
CRISPR/Cas9sgRNA1 off-target sites

| | | | No. | |
|---|---|---|---|---|
| | | Sequence of | of | Pre- |
| | | the putative | mis- | sence |
| putative | Putative | off-target | mat- | of |
| off-target | off-target | site*/SEQ ID | ching | Muta- |
| site | locus | NO: 46-50 | bases | tions |
| Cucsa.300080 | scaffold02925: | CAAAACCGGAGAA | 4 | 0 |
| | +101443 | GACGTGACGG | | |
| Intergenic | scaffold00919: | CATATGCCTAGAG | 4 | 0 |
| | 1885568 | TACGTGGGGG | | |
| Intergenic | scaffold00995: | CAAAACCCTAGAG | 3 | 0 |
| | 4267 | GGTTTGGGGG | | |
| Intergenic | scaffold02925: | CAAAACGCTAGAT | 4 | 0 |
| | +41731 | GTCTTGGAGG | | |
| Intergenic | scaffold03356: | CAAAATACTAGAG | 4 | 0 |
| | +3916928 | GACGGTGTGG | | |

* The PAM motif marked in underlined letters and mismatching bases by red letters

The transgenic lines were genotyped for indel polymorphisms using primers flanking the sgRNA1 or sgRNA2 of eIF4E target regions (Table 1). PCR products were digested with restriction enzymes BmgBI or BglII for sgRNA1 and sgRNA2 respectively. The digested products were separated on 1.5% agarose gel and the un-digested PCR products were excised, purified and cloned into the pJET1.2/blunt (Thermo Fisher Scientific). Five colonies were sequenced to discriminate indel polymorphisms and the sequences were aligned to the intact eIF4E using ClustalW BioEdit software program (Copyright©1997-2013, Tom Hall Ibis Biosciences Carlsbad, Calif., USA).

Evaluation of indel mutations in T1, T2 and T3 progeny seedlings were performed as was described above for T0 by PCR with specific primers, restriction analysis and sequencing. Screening for mutations of 20 nt deletions were performed with specific primers (Table 1 above) flanking the 20 nt deletion. This primer can only bind to DNA having a 20 nt deletion and not a 1 nt deletion.

Inoculation of Plant with Viruses

The following viruses were used for resistance analysis: ZYMV (accession No. EF062582)(Gal-On, 2000), PRSV-W (accession No. JF737858); CVYV (accession No. AY290865)(Martínez-garcía et al., 2004); CMV Fny-strain (accession No. D10538) (Rizzo and Palukaitis, 1990) and CGMMV (accession No. KF155232) (Reingold et al., 2015). Squash (Cucurbita pepo L. 'Ma'yan) plants were used as a source of inoculum of ZYMV, PRSV and CMV. Cucumber ‘Bet Alfa’ was used as a source of inoculum of CVYV and CGMMV. Cucumber seedlings at the cotyledon stage with small true leaves (about 3-5 days post emergence) were dusted with carborundum (320 mesh grit powder, Fisher Scientific, USA) prior to mechanical inoculation with virus-bearing sap (ca. 1:10 ratio g tissue/H₂O) of ZYMV, PRSV-W, CMV and CGMMV. CVYV inoculation was performed with whiteflies (Bemisia tabaci) exposed for 24 h acquisition access on CVYV-infected cucumber leaves followed by 24-h inoculation of cucumber seedlings with one true leaf (more than 10 whiteflies per seedling). Aphid inoculation of cucumber with ZYMV was performed with Aphis gossypii according to (Gal-On et al., 1992) with 5-7 aphids per plant.

Evaluation of Virus Resistance

The response of the tested plants to virus infection was determined by visual monitoring of symptoms from 28-45 days post-inoculation following RT-PCR for the presence of viral RNA.

Virus accumulation was determined by RT-PCR and Real-Time Quantitative RT-PCR (qRT-PCR). RNA samples were collected from the second and third leaves of cucumber (2 leaf discs per plant). Total RNA was extracted by TRI-REAGENT kit (Molecular Research Center, Inc., Cincinnati, Ohio, USA) and adjusted to the same concentration prior to the RT-PCR, measured by a NanoDrop ND1000 spectrophotometer (Thermo Scientific, DE, USA). First-strand cDNA was synthesized from 2 μg of total RNA using a Verso™ cDNA Kit (Thermo Fisher Scientific, Epsom, UK) with Oligo(dT) primer (100 pmol) for ZYMV-, PRSV-W- and CVYV-inoculated plants and specific virus reverse primers for CMV and CGMMV analysis.

PCR conditions were 2 min at 94° C., then 30 cycles of 30 s each at 94° C., 58° C. and 72° C., and a final elongation step of 5 min at 72° C. qPCR reactions were performed in a volume of 15 μl with 4 μl of diluted cDNA (1/4), 3 pmol of each primer and 7.5 μl Absolute QPCR SYBR Green Mix (Thermo Scientific, DE, USA). Quantitative analysis was performed using Rotor-Gene 3000 (Qiagen, Md., USA) with PCR conditions of 20 min at 95° C. (“hot start”) followed by 40 cycles of 15 s at 96° C., 15 s at 60° C., and 15 s at 72° C. The relative expression level of gene accumulation was calculated using the ΔΔCt method normalized to the reference genes using Rotor Gene Series 3000 software version 1.7.

### Example 1

### Efficacy of CRISPR/Cas9 in to Generation

To test the efficacy of CRISPR/Cas9 in cucumber and develop a new strategy to generate virus resistance, the eIF4E was disrupted. eIF4E is a plant cellular translation factor essential for the Potyviridae life cycle, and natural point mutations in this gene can confer resistance to potyviruses (for review, see (Diaz-Pendon et al., 2004; Kang et al., 2005; Sanfaçon, 2015; Le Gall et al., 2011). In cucumber, two eIF4E genes have been identified, eIF4E (accession no. XM_004147349) (236 amino acids) and eIF(iso)4E (accession no. XM_004147116.2) (204 amino acids), which share 56% nucleotide and 60% amino acid homology, respectively. Two regions in the cucumber eIF4E gene were targeted by Cas9/sgRNA, which have no homology in the eIF(iso)4E gene. The Cas9/sgRNA1 construct was designed to target the sequence in the first exon of eIF4E (positions 65-86 in the coding region) (FIG. 2A). The Cas9/sgRNA2 construct was designed to target the third exon (positions 517-540) in the coding region to allow translation of approximately two-thirds of the protein (FIG. 2A).

Five independent T0 transgenic lines were generated by Agrobacterium-mediated transformation. The presence of the transgene (Cas9/sgRNA) was confirmed by kanamycin resistance and PCR using sgRNA specific primers (FIG. 2B and Table 2 above). Three lines (1, 3 and 4) were identified as transgenic with Cas9/sgRNA1. To evaluate the types of mutations generated in sgRNA1 transgenic plants, PCR was performed in T0 plants using primers flanking the sgRNA1 target and subsequently digested with BmgBI (A site that will disappear if CAS9 and NHEJ were active in this site). In line 1, a distinct undigested fragment was observed following BmgBI restriction (FIG. 2B). The partial digestion observed indicated a heterozygous genome with both wild-type and mutant eIF4E alleles. Cloning and sequencing of the uncut BmgBI fragment showed two types of mutations, a 20 nt deletion around the PAM sequence in 7 colonies and 1 nt deletion 3 bp upstream of the PAM sequence in two colonies (FIG. 2C, SEQ ID NOS: 257-260). Whereas in line 4 (FIG. 2B) and 3 (data not shown) the amplified PCR was completely digested by BmgBI.

Two additional Cas9/sgRNA2 transgenic plants (2 and 5) did not show genome editing in T0 as determined by PCR and restriction analysis with BglII (FIG. 1b and data not shown). The study was continued with two sgRNA1 lines (1 and 4) designated CEC1-1 and CEC1-4, respectively (Cas/sgRNA1-eIF4E-Cucumber), and one sgRNA2 line (no. 5) designated CEC2-5.

### Example 2

### Genotypes and Segregation of T1 Mutants

For propagation by seeds, the Cucumis sativus CEC1-1 T0-mutant plant (derived from ‘Ilan’, a multi-pistillate, parthenocarpic greenhouse cucumber) was cross-pollinated with ‘Bet Alfa’, a monoecious, non-parthenocarpic, field cucumber. Indel polymorphisms were genotyped by PCR restriction analysis with BmgBI of the eIF4E gene in representative CEC1-1 T1 plants (FIG. 3A-B). The T1 progeny segregated into three groups (FIG. 3A-B): (a) heterozygous plants that contained about equal amounts of undigested and digested DNA (plants 5, 8, 12, 16); (b) plants with undigested DNA intensity stronger than digested DNA intensity (plants 2, 9, 7, 20); (c) non-mutants (wild type), with most of the DNA digested (plants 3, 10). The intense undigested band in group b and the faint undigested band of plant 10 might be due to the continuing activity of the cas9-sgRNA1 in transgenic plants (FIG. 3A).

The segregation of transgenic to non-transgenic in the T1 population was approximately 1:1, as expected for a single transgene locus. The independent segregation of the transgene Cas9/sgRNA1 and the eIF4E mutation indicated that they are present on different chromosomes (FIG. 3B bottom panel), allowing selection for non-transgenic mutants in later generations. To evaluate the types of mutations generated in CEC1-1 T1 plants, four representative plants (i.e. nos. 1, 4, 7 non-transgenic lacking Cas9 transgene) and no. 5 (transgenic) were chosen and the undigested DNA was cloned and sequenced. Plant no. 1 had a 20 nt deletion and plant nos. 4 and 5 had 1 nt deletions (FIG. 3B). Plant no. 7 had both, the 20 nt and 1 nt deletions as observed in the T0 (FIG. 2C). Hence, CRISPR/Cas9-induced mutations in cucumber can be stably transmitted through the germ line. PCR genotyping of the T1 generation of CEC1-1 (see Material and Methods) indicated that 20 plants had lengthy deletions (20 nt) and thirteen plants had a 1 nt deletion (data not shown).

The non-transgenic CEC1 T1 plant no. 7 (CEC1-1-7) was grown to produce seeds for the production of homozygous eIF4E mutant alleles. The all-pistillate CEC1-1-7 plant was cross-pollinated once again with the monoecious ‘Bet Alfa’. The resulting T2 progeny was genotyped and plants hemizygous for a 20 nt deletion (plant no. 1: FIG. 3B) (CEC1-1-7-1) and 1 nt deletion (plant no. 4; FIG. 3B) (CEC1-1-7-4) were self-pollinated to obtain a T3 generation. In the T3 generation, the 20 nt deletion segregated in a Mendelian manner 1:2:1 (homozygous: heterozygous: wild-type without mutation). The homozygous, non-transgenic T3 plant designated CEC1-1-7-1, heterozygous, and non-mutant (wt) plants were tested for virus resistance. Mutations in the T3 generation were confirmed by restriction analysis and sequencing.

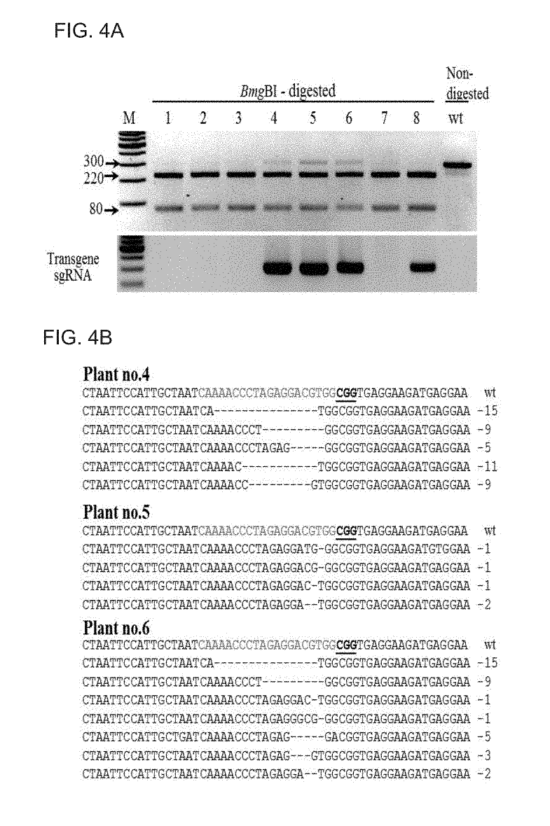

In addition to the derivatives of line no. 1 (CEC1-1), Cas9/sgRNA1-mediated mutations were analyzed in the derivatives of line no. 4 (CEC1-4) (FIGS. 2B and 3A-B). In this CEC1-4 T0 transgenic line, an indel mutation was not observed. To further confirm transgene inheritance, the plant CEC1-4 was self-pollinated and in the T1 generation, only 3 out of 8 plants (FIG. 3, plant nos. 4, 5, 6) had a faint undigested band upon digestion with BmgBI (FIG. 4A). Cloning and sequencing of the undigested faint band showed multiple mutations within the target gene in the same plant (FIG. 4B). All three plants seemed to be chimeric for the mutant allele with more than two types of mutations in the same plant. To verify whether CRISPR/Cas9 functions in the germ cell line, the CEC1-4 plants 4, 5 and 6 were grown and self-pollinated to produce a T2 generation. In the T2 generation 74 progeny plants were grown; PCR-genotyping analysis showed that most of the plants had a faint undigested band, as was observed in the T1 generation (CEC1-4). Cloning and sequencing of two representative plants from the T2 generation showed that multiple mutations were present in the same plant (data not shown). Further analysis with CEC1-4-4,5,6 plants was not done because it appeared that in this CEC1-4 line Cas9/sgRNA1 mediated cleavage occurred in somatic cells, and not in the germ line.

To evaluate the types of mutations mediated by Cas9/sgRNA2 in line CEC2-5, the flanking region was PCR-amplified and the presence of indel mutations was tested by BglII restriction (FIG. 2B). The PCR fragment of CEC2-5 was as completely digested as the wild-type (FIG. 2B). CEC2-5 was cross pollinated with ‘Bet Alfa’ and the progeny (T1) segregated to approximately 1:1 transgenic: non-transgenic, suggesting a single transgenic locus. Mutations were observed only in transgenic progeny (FIG. 5A). Genotyping of CEC2-5 T1-generation plants revealed three groups: a. Homozygous plants with completely undigested DNA (9 out of 15 transgenic seedlings) (FIG. 5A, plants 6 and 14); b. Heterozygous plants having similar intensities of undigested and digested DNA (FIG. 5A, plants 9 and 26); c. Plants where faintly digested DNA can be seen, which may reflect continuing activity of Cas9/sgRNA2 in heterozygous plants (FIG. 5A, plants 1 and 7). The DNA of non-transgenic segregant plants lacking Cas9 was completely digested, as was the wild-type non-transgenic control (FIG. 5A, plants 2 and 3).

To genotype the mutations in CEC2 T1 progeny plants, the complete uncut PCR product was cloned and sequenced from four representative plants (FIG. 5B, plants 6, 14, 15, 18). The results showed that each plant had two different types of mutations (FIG. 5B). Interestingly, in plant 6 an insertion of one nucleotide along with a two nucleotide deletion was found. All of the plants had bi-allelic mutations, but the mutations were different from plant to plant. Mutations were found only in the T1 transgenic plants but not in T0 plants. This suggests that editing occurred in the germ cell line in the T0 generation.

Bi-allelic heterozygous mutant and mono-allelic mutant T1 plants of CEC2-5-1 were cross-pollinated with non-transgenic ‘Bet Alfa’ plants and the resulting T2 progeny seeds were pooled (designated as Mix (M) CEC2-5-M) and germinated. The mutant plants were screened by PCR/BglII restriction analysis. To obtain homozygous mutant plants of CEC2-5-M, T2 homozygous CEC2-5-M-9, CEC2-5-M-16 and heterozygous CEC2-5-M-8, CEC2-5-M-21 seedlings were cross-pollinated with T2 plants. Sequencing of T3 progenies of plants 9, 16, 8 and 21 showed 4 nt deletions. The T3 progenies of plants 9, 16, 8 and 21 were used for virus resistance analysis.

### Example 3

### Off-Target Analysis

Cas9/sgRNA1 off-targets were evaluated by the CRISPR-P program (Lei et al., 2014) using the sgRNA1 sequence against the cucumber genome. Five candidate potential off-targets were determined (Table 2 above). PCR and sequencing of these candidate targets revealed no changes in the genome of non-transgenic T3 generation CEC1-1-7-1.

### Example 4

### Virus Resistance Analysis

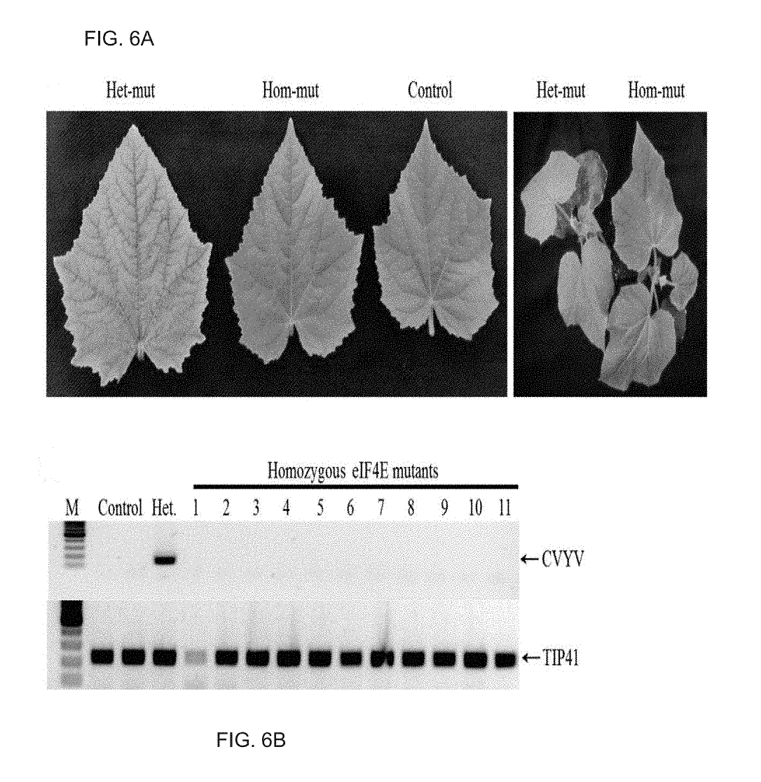

To test whether CRISPR/Cas9-mediated mutations in eIF4E confer virus resistance, T3 progenies of CEC1 and CEC2 seedlings were inoculated with CVYV-ipomovirus, two potyviruses ZYMV and PRSV-W and Cucumber mosaic cucumovirus (CMV) and Cucumber green mottle mosaic tobamovirus (CGMMV). T3 non-transgenic progenies of CEC1-1-7-1 showed a Mendelian segregation ratio of 1:2:1 for the homozygous mutant allele (eif4e), heterozygote mutant allele and homozygous non-mutant. In the case of CEC2-5-M-9, 16, 8, 21 (mixture of 4 lines: 4n) (designated CEC2-5-M-4n), the T3 progenies of plants 9 and 16 segregated 1:1 (homozygous: heterozygous) and plants 8 and 21 segregated 1:2:1 (homozygous: heterozygous: non-mutant). Whitefly inoculation (natural vector) of the T3 generation with CVYV showed that both CEC1-1-7-1 and CEC2-5-M-4n homozygous mutant plants were immune to CVYV infection (0/20 and 0/32, respectively), whereas in the heterozygous mutant and wild type plants severe symptoms were observed 7-10 days post infection (dpi) (Table 3, below, and FIG. 6A).

TABLE 3
Response of T3 generation plants of non-transgenic CEC-
1-7-1 and CEC2-5-M-4n lines to CVYV, ZYMV, PRSV-W, CMV
and CGMMV infection at different days post infection (dpi).

CEC1-1-7-1

| | |
|---|---|
| Homozygous^{□} | Non-homozygous^{§} |

| Virus | 14 dpi | 25-45 dpi* | 14 dpi |
|---|---|---|---|
| CVYV | 0/20 | 0/12 | 60/60 |
| ZYMV | 0/28 | 10/21 | 97/97 |
| PRSV | 1/14 | n.t^{⋄} | 40/41 |
| CMV | 11/11 | — | 9/9 |
| CGMMV | 10/10 | — | 10/10 |

CEC2-5-M-4n

| | |
|---|---|
| Homozygous | non-homozygous |

| | | | |
|---|---|---|---|
| CVYV | 0/32 | 0/10 | 42/43 |
| ZYMV | 0/64 | 16/63 | 67/69 |
| ZYMV° | 0/8 | 0/8 | 6/7 |
| PRSV | 1/55 | 7/18 | 33/37 |

^{□}Infectivity rates were scored as number of symptomatic plants of the total number of plants inoculated.
^{§}Non-homozygous plants include heterozygous and non-mutant plants.
*Some of the resistant plants were kept in a net house for further observation.
^{⋄}n.t = not tested.
°Plants were inoculated by 5-10 aphids per plant.

The homozygous mutant plants tested for infectivity after 45 dpi. The experiment was repeated 4 times with consistent results (Table 3). RT-PCR analyses revealed no viral RNA accumulation in the homozygous mutant plants, whereas in the heterozygous plants viral RNA accumulation was observed in similar quantities as the wild-type (FIG. 6B).

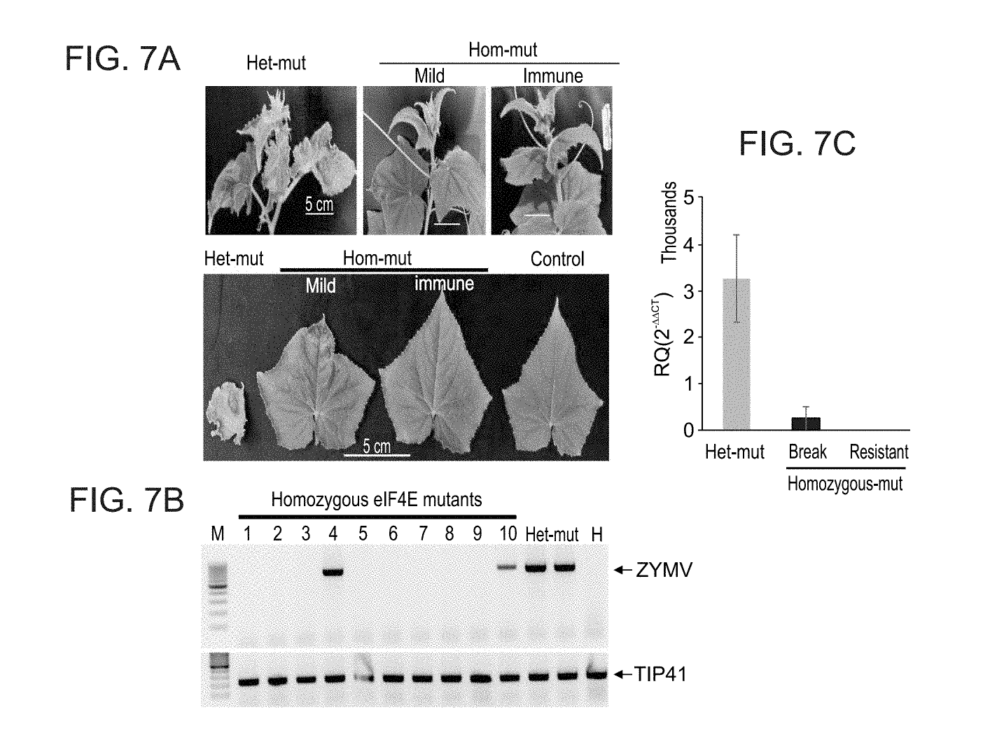

Following ZYMV inoculation (mechanical and by aphids) of CEC1-1-7-1 and CEC2-5-M-4n the T3 lines, seedlings showed mosaic symptom development 7-10 dpi that exacerbated to severe symptoms of leaf deformation and stunting 20 dpi in heterozygous and non-mutant plants (FIG. 7A), whereas, the eif4e mutant plants did not display disease symptoms 20 dpi (FIG. 7A). Accordingly, resistance to ZYMV systemic infection was observed (in 0 of 28 plants infected) in four separate biological repeat experiments (Table 3 above). However, 25-45 dpi mild symptoms could be observed in 25-48% of the T3 homozygous plants in three biological experiments with CEC1-1-7-1 (10 out of 21 plants) and CEC2-5-M-4n (16 out of 63 plants) (Table 2).

However, the mild symptoms appeared only in patches (FIG. 7A) and the plants developed normally similar to the non-infected plants, compared to the stunted plants with deformed fruit of the infected heterozygous and wild-type plants. ZYMV RNA was not detected in the immune homozygous resistant plants (FIG. 7B). The late appearance of mild symptoms was accompanied by accumulation of ZYMV RNA in the upper leaves (plants 4 and 10, FIG. 7B). The level of ZYMV RNA accumulation in mild symptomatic plants was lower in CEC1-1-7-1 and CEC2-5-M-21 homozygous plants than in the wild type (FIG. 7C). Interestingly, resistance breaking was not observed when inoculation was made by the natural vector of ZYMV, Aphis gossypii (Table 3 above).

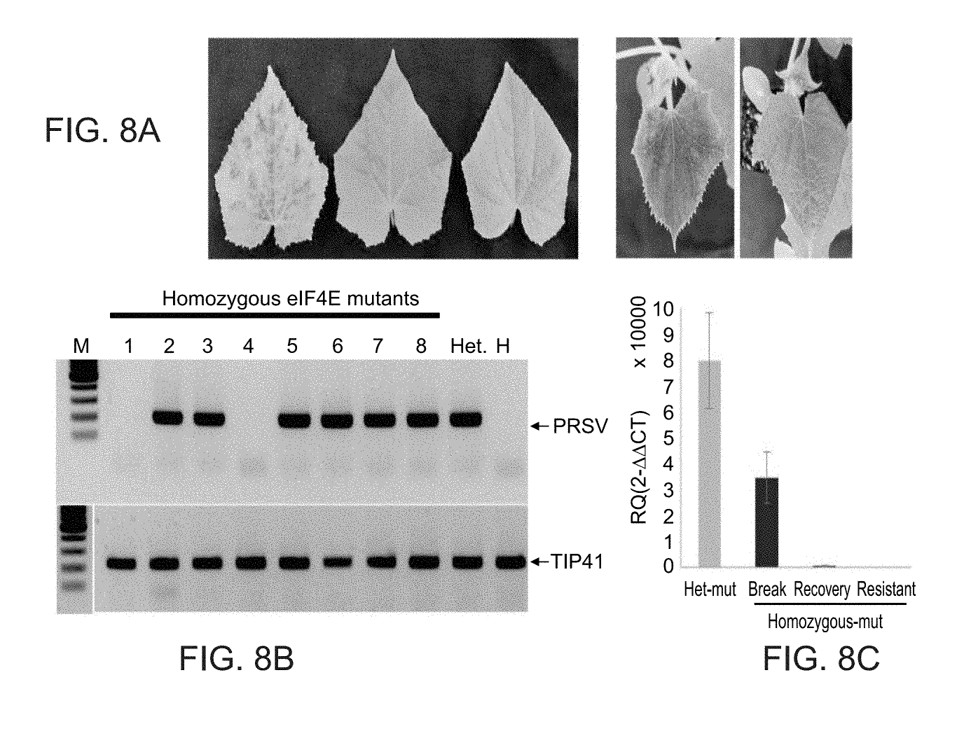

Resistance to PRSV-W (Israeli isolate) was assessed following mechanical inoculation of T3 generation seedlings of CEC1-1-7-1 and CEC2-5-M-4n. PRSV-W symptoms in wild-type cucumber were less aggressive than ZYMV, and severe symptoms appeared 14 dpi. Resistance to PRSV-W can be seen in CEC1-1-7-1 (FIG. 8A) and CEC2-5-M-4n at 14 dpi (data not shown). In about 40% of the resistant eif4e plants, mild symptoms appeared 21 dpi (Table 3 above), although such resistance breaking did not affect plant development. In most of the asymptomatic resistant plants, PRSV-W RNA accumulation was detected (FIG. 8B), however, its RNA levels were significantly lower than in the infected heterozygous plants (FIG. 8C). A recovery phenomenon was observed in 4 out of 7 resistance-breaking plants together with a significant decrease of viral RNA level 35 dpi.

CEC1-1-7-1 T3 progenies were tested for resistance to CMV (Cucumovirus) and CGMMV (Tobamovirus) as these viruses have 5′ capped RNA. Virus symptoms were observed in all plants (homozygous mutants, heterozygous mutants and wild type) (Table 2) without significant differences in symptom appearance. The level of CMV and CGMMV RNAs between the mutants was not tested.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Other References are Cited Throughout the Application

Abdul-Razzak, A., Guiraud, T., Peypelut, M., Walter, J., Houvenaghel, M.-C., Candresse, T., Le Gall, O. and German-Retana, S. (2009) Involvement of the cylindrical inclusion (CI) protein in the overcoming of an eIF4E-mediated resistance against Lettuce mosaic potyvirus. Mol. Plant Pathol. 10, 109-113.
Ayme, V., Souche, S., Caranta, C., Jacquemond, M., Chadceuf, J., Palloix, A. and Moury, B. (2006) Different Mutations in the Genome-Linked Protein VPg of Potato virus Y Confer Virulence on the pvr23 Resistance in Pepper. Mol. Plant. Microbe Interact. 19, 557-563.
Belhaj, K., Chaparro-Garcia, A., Kamoun, S. and Nekrasov, V. (2013) Plant genome editing made easy: targeted mutagenesis in model and crop plants using the CRISPR/Cas system. Plant Methods, 9, 39.
Belhaj, K., Chaparro-Garcia, A., Kamoun, S., Patron, N. J. and Nekrasov, V. (2015) Editing plant genomes with CRISPR/Cas9. Curr. Opin. Biotechnol. 32, 76-84.
Bortesi, L. and Fischer, R. (2015) The CRISPR/Cas9 system for plant genome editing and beyond. Biotechnol. Adv. 33, 41-52.
Brooks, C., Nekrasov, V., Lippman, Z. B. and Eck, J. V. (2014) Efficient Gene Editing in Tomato in the First Generation Using the Clustered Regularly Interspaced Short Palindromic Repeats/CRISPR-Associated9 System. Plant Physiol. 166, 1292-1297.
Dafny-Yelin, M. and Tzfira, T. (2007) Delivery of Multiple Transgenes to Plant Cells. Plant Physiol. 145, 1118-1128.
Dellaporta, S. L., Wood, J. and Hicks, J. B. (1983) A plant DNA minipreparation: Version II. Plant Mol. Biol. Report. 1, 19-21.
Diaz-Pendon, J. A., Truniger, V., Nieto, C., Garcia-Mas, J., Bendahmane, A. and Aranda, M. A. (2004) Advances in understanding recessive resistance to plant viruses. Mol. Plant Pathol. 5, 223-233.
Duprat, A., Caranta, C., Revers, F., Menand, B., Browning, K. S. and Robaglia, C. (2002) The Arabidopsis eukaryotic initiation factor (iso)4E is dispensable for plant growth but required for susceptibility to potyviruses. Plant J. 32, 927-934.
Feng, Z., Zhang, B., Ding, W., Liu, X., Yang, D.-L., Wei, P., Cao, F., Zhu, S., Zhang, F., Mao, Y. and Zhu, J.-K. (2013) Efficient genome editing in plants using a CRISPR/Cas system. Cell Res. 23, 1229-1232.
Fu, Y., Foden, J. A., Khayter, C., Maeder, M. L., Reyon, D., Joung, J. K. and Sander, J. D. (2013) High-frequency off-target mutagenesis induced by CRISPR-Cas nucleases in human cells. Nat. Biotechnol. 31, 822-826.
Gal-On, A. (2000) A Point Mutation in the FRNK Motif of the Potyvirus Helper Component-Protease Gene Alters Symptom Expression in Cucurbits and Elicits Protection Against the Severe Homologous Virus. Phytopathology, 90, 467-473.
Gal-On, A., Antignus, Y., Rosner, A. and Raccah, B. (1992) A zucchini yellow mosaic virus coat protein gene mutation restores aphid transmissibility but has no effect on multiplication. J. Gen. Virol. 73, 2183-2187.
Gal-On, A., Wolf, D., Antignus, Y., Paths, L., Ryu, K. H., Min, B. E., Pearlsman, M., Lachman, O., Gaba, V., Wang, Y., Shiboleth, Y. M., Yang, J. and Zelcer, A. (2005) Transgenic cucumbers harboring the 54-kDa putative gene of Cucumber fruit mottle mosaic tobamovirus are highly resistant to viral infection and protect non-transgenic scions from soil infection. Trans genic Res. 14, 81-93.
Gómez, P., Rodríguez-Hernández, A. M., Moury, B. and Aranda, M. A. (2009) Genetic resistance for the sustainable control of plant virus diseases: breeding, mechanisms and durability. Eur. J. Plant Pathol. 125, 1-22.
Hébrard, E., Pinel-Galzi, A., Bersoult, A., Sire, C. and Fargette, D. (2006) Emergence of a resistance-breaking isolate of Rice yellow mottle virus during serial inoculations is due to a single substitution in the genome-linked viral protein VPg. J. Gen. Virol. 87, 1369-1373.
Hwang, J., Li, J., Liu, W.-Y., An, S.-J., Cho, H., Her, N. H., Yeam, I., Kim, D. and Kang, B.-C. (2009) Double mutations in eIF4E and eIFiso4E confer recessive resistance to Chilli veinal mottle virus in pepper. Mol. Cells. 27, 329-336.
Jackson, R. J., Hellen, C. U. T. and Pestova, T. V. (2010) The mechanism of eukaryotic translation initiation and principles of its regulation. Nat. Rev. Mol. Cell Biol. 11, 113-127.
Jacobs, T. B., LaFayette, P. R., Schmitz, R. J. and Parrott, W. A. (2015) Targeted genome modifications in soybean with CRISPR/Cas9. BMC Biotechnol. 15, 16-26.
Jiang, J. and Laliberté, J.-F. (2011) The genome-linked protein VPg of plant viruses—a protein with many partners. Curr. Opin. Virol. 1, 347-354.
Jones, H. D. (2015) Regulatory uncertainty over genome editing. Nat. Plants. 1, 14011. Kang, B.-C., Yeam, I. and Jahn, M. M. (2005) Genetics of Plant Virus Resistance. Annu. Rev. Phytopathol. 43, 581-621.
Le Gall, O., Aranda, M. A. and Caranta, C. (2011) Plant resistance to viruses mediated by translation initiation factors. In C. Caranta, M. A. Aranda, M. Tepfer, and J. Lopez-Moya, eds. In Recent Advances in Plant Virology, Academic Press, pp. 177-194.
Lei, Y., Lu, L., Liu, H.-Y., Li, S., Xing, F. and Chen, L.-L. (2014) CRISPR-P: a web tool for synthetic single-guide RNA design of CRISPR-system in plants. Mol. Plant. 7, 1494-1496.
Lellis, A. D., Kasschau, K. D., Whitham, S. A. and Carrington, J. C. (2002) Loss-of-Susceptibility Mutants of Arabidopsis thaliana Reveal an Essential Role for eIF(iso)4E during Potyvirus Infection. Curr. Biol. 12, 1046-1051.
Léonard, S., Plante, D., Wittmann, S., Daigneault, N., Fortin, M. G. and Laliberté, J.-F. (2000) Complex Formation between Potyvirus VPg and Translation Eukaryotic Initiation Factor 4E Correlates with Virus Infectivity. J. Virol. 74, 7730-7737.
Li, J.-F., Norville, J. E., Aach, J., McCormack, M., Zhang, D., Bush, J., Church, G. M. and Sheen, J. (2013) Multiplex and homologous recombination-mediated genome editing in Arabidopsis and Nicotiana benthamiana using guide RNA and Cas9. Nat. Biotechnol. 31, 688-691.
Ling, K.-S., Harris, K. R., Meyer, J. D. F., Levi, A., Guner, N., Wehner, T. C., Bendahmane, A. and Havey, M. J. (2009) Non-synonymous single nucleotide polymorphisms in the watermelon eIF4E gene are closely associated with resistance to Zucchini yellow mosaic virus. Theor. Appl. Genet. 120, 191-200.
Liu, L. and Fan, X.-D. (2014) CRISPR-Cas system: a powerful tool for genome engineering. Plant Mol. Biol. 85, 209-218.
Mali, P., Aach, J., Stranges, P. B., Esvelt, K. M., Moosburner, M., Kosuri, S., Yang, L. and Church, G. M. (2013) CAS9 transcriptional activators for target specificity screening and paired nickases for cooperative genome engineering. Nat. Biotechnol. 31, 833-838.
Martínez-garcía, B., Marco, C. F., Goytia, E., López-abella, D., Serra, M. T., Aranda, M. A. and López-moya, J. J. (2004) Development and use of detection methods specific for Cucumber vein yellowing virus (CVYV). Eur. J. Plant Pathol. 110, 811-821.
Maule, A. J., Caranta, C. and Boulton, M. I. (2007) Sources of natural resistance to plant viruses: status and prospects. Mol. Plant Pathol. 8, 223-231.
Mazier, M., Flamain, F., Nicolaï, M., Sarnette, V. and Caranta, C. (2011) Knock-Down of Both eIF4E1 and eIF4E2 Genes Confers Broad-Spectrum Resistance against Potyviruses in Tomato. PLoS ONE, 6, e29595.
Moury, B., Fabre, F. and Senoussi, R. (2007) Estimation of the number of virus particles transmitted by an insect vector. Proc. Natl. Acad. Sci. 104, 17891-17896.
Moury, B., Morel, C., Johansen, E., Guilbaud, L., Souche, S., Ayme, V., Caranta, C., Palloix, A. and Jacquemond, M. (2004) Mutations in Potato virus Y Genome-Linked Protein Determine Virulence Toward Recessive Resistances in Capsicum annuum and Lycopersicon hirsutum. Mol. Plant. Microbe Interact. 17, 322-329.
Nakahara, K. S., Shimada, R., Choi, S.-H., Yamamoto, H., Shao, J. and Uyeda, I. (2010) Involvement of the P1 Cistron in Overcoming eIF4E-Mediated Recessive Resistance Against Clover yellow vein virus in Pea. Mol. Plant. Microbe Interact. 23, 1460-1469.
Nekrasov, V., Staskawicz, B., Weigel, D., Jones, J. D. G. and Kamoun, S. (2013) Targeted mutagenesis in the model plant Nicotiana benthamiana using Cas9 RNA-guided endonuclease. Nat. Biotechnol. 31, 691-693.
Nicaise, V., German-Retana, S., Sanjuán, R., Dubrana, M.-P., Mazier, M., Maisonneuve, B., Candresse, T., Caranta, C. and LeGall, O. (2003) The Eukaryotic Translation Initiation Factor 4E Controls Lettuce Susceptibility to the Potyvirus Lettuce mosaic virus. Plant Physiol. 132, 1272-1282.
Piron, F., Nicolaï, M., Minoïa., S., Piednoir, E., Moretti, A., Salgues, A., Zamir, D., Caranta, C. and Bendahmane, A. (2010) An Induced Mutation in Tomato eIF4E Leads to Immunity to Two Potyviruses. PLoS ONE, 5, e11313.
Reingold, V., Lachman, O., Blaosov, E. and Dombrovsky, A. (2015) Seed disinfection treatments do not sufficiently eliminate the infectivity of Cucumber green mottle mosaic virus (CGMMV) on cucurbit seeds. Plant Pathol. 64, 245-255.
Revers, F. and García, J. A. (2015) Chapter Three—Molecular Biology of Potyviruses. In K. M. and T. C. Mettenleiter, ed. Advances in Virus Research. Academic Press, pp. 101-199.
Rizzo, T. M. and Palukaitis, P. (1990) Construction of full-length cDNA clones of cucumber mosaic virus RNAs 1, 2 and 3: generation of infectious RNA transcripts. Mol. Gen. Genet. 222, 249-256.
Rodríguez-Hernández, A. M., Gosalvez, B., Sempere, R. N., Burgos, L., Aranda, M. A. and Truniger, V. (2012) Melon RNA interference (RNAi) lines silenced for Cm-eIF4E show broad virus resistance. Mol. Plant Pathol. 13, 755-763.
Ruffel, S., Dussault, M.-H., Palloix, A., Moury, B., Bendahmane, A., Robaglia, C. and Caranta, C. (2002) A natural recessive resistance gene against potato virus Y in pepper corresponds to the eukaryotic initiation factor 4E (eIF4E). Plant J. 32, 1067-1075.
Ruffel, S., Gallois, J.-L., Moury, B., Robaglia, C., Palloix, A. and Caranta, C. (2006) Simultaneous mutations in translation initiation factors eIF4E and eIF(iso)4E are required to prevent pepper veinal mottle virus infection of pepper. J. Gen. Virol. 87, 2089-2098.
Sacristán, S., Malpica, J. M., Fraile, A. and García-Arenal, F. (2003) Estimation of Population Bottlenecks during Systemic Movement of Tobacco Mosaic Virus in Tobacco Plants. J. Virol. 77, 9906-9911.
Sanfaçon, H. (2015) Plant Translation Factors and Virus Resistance. Viruses, 7, 3392-3419.
Sato, M., Nakahara, K., Yoshii, M., Ishikawa, M. and Uyeda, I. (2005) Selective involvement of members of the eukaryotic initiation factor 4E family in the infection of Arabidopsis thaliana by potyviruses. FEBS Lett. 579, 1167-1171.
Sternberg, S. H., Redding, S., Jinek, M., Greene, E. C. and Doudna, J. A. (2014) DNA interrogation by the CRISPR RNA-guided endonuclease Cas9. Nature, 507, 62-67.
Truniger, V. and Aranda, M. A. (2009) Recessive Resistance to Plant Viruses. In Gad Loebenstein and John P. Carr, ed. Advances in Virus Research. Caister Academic Press: Wymondham, Norfolk, Va., USA, pp. 119-231.
Wang, A. and Krishnaswamy, S. (2012) Eukaryotic translation initiation factor 4E-mediated recessive resistance to plant viruses and its utility in crop improvement. Mol. Plant Pathol. 13, 795-803.
Wittmann, S., Chatel, H., Fortin, M. G. and Laliberté, J.-F. (1997) Interaction of the Viral Protein Genome Linked of Turnip Mosaic Potyvirus with the Translational Eukaryotic Initiation Factor (iso) 4E of Arabidopsis thaliana Using the Yeast Two-Hybrid System. Virology, 234, 84-92.
Xie, K., Zhang, J. and Yang, Y. (2014) Genome-Wide Prediction of Highly Specific Guide RNA Spacers for CRISPR-Cas9-Mediated Genome Editing in Model Plants and Major Crops. Mol. Plant. 7, 923-926.
Xu, R.-F., Li, H., Qin, R.-Y., Li, J., Qiu, C.-H., Yang, Y.-C., Ma, H., Li, L., Wei, P.-C.
and Yang, J.-B. (2015) Generation of inheritable and “transgene clean” targeted genome-modified rice in later generations using the CRISPR/Cas9 system. Sci. Rep. 5, 11491.
Zhang, H., Zhang, J., Wei, P., Zhang, B., Gou, F., Feng, Z., Mao, Y., Yang, L., Zhang, H., Xu, N. and Zhu, J.-K. (2014) The CRISPR/Cas9 system produces specific and homozygous targeted gene editing in rice in one generation. Plant Biotechnol. J. 12, 797-807.

1. A cucumber plant comprising a genome being homozygous for a loss of function mutation in an eIF4E gene.

2. A method of increasing viral resistance in a cucumber plant, the method comprising subjecting a genome of the cucumber plant to a DNA editing agent so as to induce a loss of function mutation in an eIF4E gene so as to impart recessive resistance resultant of said loss of function mutation.

3. The method of claim 2, further comprising subjecting the cucumber plant comprising said loss of function in said eIF4E gene to at least one step of crossing or selfing.

4. The plant of claim 1, comprising a heterologous nucleic acid sequence encoding an endonuclease of a genome editing agent.

5. The plant of claim 1, said cucumber plant exhibiting higher resistance to an RNA virus as compared to that of a wild-type cucumber plant of the same genetic background.

6. The plant of claim 5, wherein said RNA virus comprises a plurality of viruses comprising CVYV, ZYMV and PRSV-W.

7. The method of claim 2, wherein said RNA virus is an uncapped virus having the VPg protein covalently linked to the viral RNA 5′.

8. The method of claim 2, wherein said virus belongs to the potyviridae family.

9. (canceled)

10. The plant of claim 1, wherein said resistance is manifested by absence, delayed or milder symptoms appearance.

11. The plant of claim 5, wherein said resistance is manifested by no or reduced accumulation of RNA of said virus or by visual monitoring of symptoms.

12. The plant of claim 1, wherein said DNA editing agent is directed to exon 1 of a coding sequence of said eIF4E.

13. The plant of claim 1, wherein said DNA editing agent is directed to exon 3 of a coding sequence of said eIF4E.

14. The plant of claim 1, wherein said DNA editing agent does not edit the coding sequence of eIF(iso)4E.

15. (canceled)

16. A plant part comprising DNA of the plant of claim 1.

17. The plant part of claim 16, being a seed or a fruit.

18.-19. (canceled)

20. The method of claim 2, further comprising breeding out said DNA editing agent.

21. The method of claim 20, Further comprising selecting a cucumber plant wherein said DNA editing agent and said eIF4E gene are on different chromosomes prior to said breeding out.

22. The plant of claim 1, wherein said DNA editing agent is of a DNA editing system selected from the group consisting of meganucleases, Zinc finger nucleases (ZFNs), transcription-activator like effector nucleases (TALENs) and CRISPR.

23. The plant of claim 1, wherein said DNA editing agent is of CRISPR/Cas-9.

24. The plant of claim 1, wherein said DNA editing agent comprises a guide RNA (sgRNA) selected from the group consisting of SEQ ID NOs: 51-254 or 255-256.


SEQUENCE LISTING (300 sequences)

SEQ ID NO: 1 - DNA, 708 bp, Cucumis sativus
atggtagttgaagatacgatcaaagctacatcggcggaagatctttctaattccattgctaatcaaaaccctagaggacgtggcggtgaggaagatgaggaacttgaggaaggtgagatcgtcggcgatgacgaccttgactcctccaatttgaccgcggccttagttcatcagcctcaccctcttgagcactcttggaccttttggtttgataacccatctgccaaatccaagcaagccacctggggtgcgtctatgcgaccgatctataccttctccactgtcgaggagttctggagtgtttacaacaatattcatcatccaagcaaattggcgttgagggcagatttgtactgcttcaaacataaaattgagcctaaatgggaagatcctgtttgtgcgagtggagggaagtggactgtgaacttttcaaggggaaaatctgataatggctggttgtacacgctgcttgctatgatcggagaacagtttgactgtggtgatgaaatttgtggagcagttgttaatgttagatctgggcaggataaaatatcgatttggacaaaaaatgcttccaatgaagctgcgcaggcgagcattggaaaacagtggaaggagtttcttgattacaatgagagcattggctttatattccacgatgatgcaaagaaatttgatagacatgcgaagaataaatatatggtgtga

SEQ ID NO: 2 - DNA, 612 bp, Cucumis sativus
atggccggtgaggtagcggtggagggtgctgtggcggcggcggcgttggcgacagaagtagccgattccaatccccaggcccataaattggagagaaaatggaccttttggtttgataatcagtccaggccgaagcaaggtgctgcttggggcacctctcttcgcaaggtctataccttcgacaccgtcgaagaattttggtgtttgtacgatcagttattcaagccaagcaagttaccggcgaatgcagattttcacctgttcaaaactggagttgaaccgaaatgggaagatcccgagtgcgctaatggaggaaaatggactgtcaccagcagtcgaaaggctaaccttgataatatgtggctggaaacgttgatggccttgattggggaacaatttgaggagtctgatgagatctgtggcgtagttgccagtgtgcgccagaggcaggacaaacttgctctgtggaccaagacagcaacaaacgaggctgctcagatgagcattggtaggaaatggaaggagattatcgatgtgaccgacaagatttccttcagctttcatgaggatttgagaagggaaaagtcggcaaaagctcgatatagtgtttga

SEQ ID NO: 3 - DNA, 59 bp, Artificial sequence
  Single strand DNA oligonucleotide
agagtcgacatagcgattgaaaaccctagaggacgtgggttttagagctagaaatagca

SEQ ID NO: 4 - DNA, 20 bp, Artificial sequence
  Single strand DNA oligonucleotide
gaaaaccctagaggacgtgg

SEQ ID NO: 5 - DNA, 60 bp, Artificial sequence
  Single strand DNA oligonucleotide
agagtcgacatagcgattgcagttgttaatgttagatctgttttagagctagaaatagca

SEQ ID NO: 6 - DNA, 21 bp, Artificial sequence
  Single strand DNA oligonucleotide
gcagttgttaatgttagatct

SEQ ID NO: 7 - DNA, 26 bp, Artificial sequence
  Single strand DNA oligonucleotide
gctaagcttagaactaaaaaaagcac

SEQ ID NO: 8 - DNA, 29 bp, Artificial sequence
  Single strand DNA oligonucleotide
gctctagaatggtagttgaagatacgatc

SEQ ID NO: 9 - DNA, 29 bp, Artificial sequence
  Single strand DNA oligonucleotide
gcgcggccgctcacaccatatatttattc

SEQ ID NO: 10 - DNA, 20 bp, Artificial sequence
  Single strand DNA oligonucleotide
gcgtatccgttcttagactg

SEQ ID NO: 11 - DNA, 20 bp, Artificial sequence
  Single strand DNA oligonucleotide
ggttgtaggtctgaacaagc

SEQ ID NO: 12 - DNA, 20 bp, Artificial sequence
  Single strand DNA oligonucleotide
gacactgacggctttatgcc

SEQ ID NO: 13 - DNA, 26 bp, Artificial sequence
  Single strand DNA oligonucleotide
gtgctttttttagatctaagcttagc

SEQ ID NO: 14 - DNA, 21 bp, Artificial sequence
  Single strand DNA oligonucleotide
atggtagttgaagatacgatc

SEQ ID NO: 15 - DNA, 20 bp, Artificial sequence
  Single strand DNA oligonucleotide
ctccagaactcctcgacagt

SEQ ID NO: 16 - DNA, 20 bp, Artificial sequence
  Single strand DNA oligonucleotide
cgttgagggcagatttgtac

SEQ ID NO: 17 - DNA, 20 bp, Artificial sequence
  Single strand DNA oligonucleotide
tattcttcgcatgtctatca

SEQ ID NO: 18 - DNA, 21 bp, Artificial sequence
  Single strand DNA oligonucleotide
caaaaccctagatgaggaact

SEQ ID NO: 19 - DNA, 20 bp, Artificial sequence
  Single strand DNA oligonucleotide
ctccagaactcctcgacagt

SEQ ID NO: 20 - DNA, 20 bp, Artificial sequence
  Single strand DNA oligonucleotide
attcccaagctcagcaaaga

SEQ ID NO: 21 - DNA, 22 bp, Artificial sequence
  Single strand DNA oligonucleotide
cgaacctttctatctcccaatg

SEQ ID NO: 22 - DNA, 20 bp, Artificial sequence
  Single strand DNA oligonucleotide
acatgcatctgactggtgag

SEQ ID NO: 23 - DNA, 22 bp, Artificial sequence
  Single strand DNA oligonucleotide
agttgcaacatccatcaatgaa

SEQ ID NO: 24 - DNA, 22 bp, Artificial sequence
  Single strand DNA oligonucleotide
agctccatacatagctgagaca

SEQ ID NO: 25 - DNA, 20 bp, Artificial sequence
  Single strand DNA oligonucleotide
gaaccaagaggcgaattgct

SEQ ID NO: 26 - DNA, 22 bp, Artificial sequence
  Single strand DNA oligonucleotide
gaatggtacatcaccggacata

SEQ ID NO: 27 - DNA, 20 bp, Artificial sequence
  Single strand DNA oligonucleotide
cggagtggcatgctctatta

SEQ ID NO: 28 - DNA, 19 bp, Artificial sequence
  Single strand DNA oligonucleotide
ctgatctgggcgacaagga

SEQ ID NO: 29 - DNA, 20 bp, Artificial sequence
  Single strand DNA oligonucleotide
cgataacgacagcaaaacac

SEQ ID NO: 30 - DNA, 20 bp, Artificial sequence
  Single strand DNA oligonucleotide
gtttcgcttctcagctccac

SEQ ID NO: 31 - DNA, 20 bp, Artificial sequence
  Single strand DNA oligonucleotide
cgcgtcatcagtacgcttta

SEQ ID NO: 32 - DNA, 18 bp, Artificial sequence
  Single strand DNA oligonucleotide
ggttcatctggtggtctt

SEQ ID NO: 33 - DNA, 21 bp, Artificial sequence
  Single strand DNA oligonucleotide
ctttaaacgaacggtcagtcc

SEQ ID NO: 34 - DNA, 29 bp, Artificial sequence
  Single strand DNA oligonucleotide
caacaggtgatattggattatgattatac

SEQ ID NO: 35 - DNA, 21 bp, Artificial sequence
  Single strand DNA oligonucleotide
gccagctcatcctcatataag

SEQ ID NO: 36 - DNA, 20 bp, Artificial sequence
  Single strand DNA oligonucleotide
gaacgccctggaaaacggac

SEQ ID NO: 37 - DNA, 20 bp, Artificial sequence
  Single strand DNA oligonucleotide
ggccattggtgttttcaaac

SEQ ID NO: 38 - DNA, 23 bp, Artificial sequence
  Single strand DNA oligonucleotide
agaaggactacattattagagag

SEQ ID NO: 39 - DNA, 24 bp, Artificial sequence
  Single strand DNA oligonucleotide
tttaaagtaagatcttgaatgatc

SEQ ID NO: 40 - DNA, 20 bp, Artificial sequence
  Single strand DNA oligonucleotide
caaccaccaccaaccgacat

SEQ ID NO: 41 - DNA, 20 bp, Artificial sequence
  Single strand DNA oligonucleotide
ggatgttgagatcatcttga

SEQ ID NO: 42 - DNA, 20 bp, Artificial sequence
  Single strand DNA oligonucleotide
atgacatgcccatctcgcct

SEQ ID NO: 43 - DNA, 21 bp, Artificial sequence
  Single strand DNA oligonucleotide
cacaaggaagattaagtgaga

SEQ ID NO: 44 - DNA, 23 bp, Artificial sequence
  Single strand DNA oligonucleotide
aaagttacaaatgttggaagaca

SEQ ID NO: 45 - DNA, 20 bp, Artificial sequence
  Single strand DNA oligonucleotide
aatgagttgctactagaccc

SEQ ID NO: 46 - DNA, 23 bp, Artificial sequence
  Single strand DNA oligonucleotide
caaaaccggagaagacgtgacgg

SEQ ID NO: 47 - DNA, 23 bp, Artificial sequence
  Single strand DNA oligonucleotide
catatgcctagagtacgtggggg

SEQ ID NO: 48 - DNA, 23 bp, Artificial sequence
  Single strand DNA oligonucleotide
caaaaccctagagggtttggggg

SEQ ID NO: 49 - DNA, 23 bp, Artificial sequence
  Single strand DNA oligonucleotide
caaaacgctagatgtcttggagg

SEQ ID NO: 50 - DNA, 23 bp, Artificial sequence
  Single strand DNA oligonucleotide
caaaatactagaggacggtgtgg

SEQ ID NO: 51 - DNA, 23 bp, Artificial sequence
  a guide RNA (sgRNA) nucleic acid sequence
cggtcgcatagacgcaccccagg

SEQ ID NO: 52 - DNA, 23 bp, Artificial sequence
  a guide RNA (sgRNA) nucleic acid sequence
cgcaccccaggtggcttgcttgg

SEQ ID NO: 53 - DNA, 23 bp, Artificial sequence
  a guide RNA (sgRNA) nucleic acid sequence
tcgcatagacgcaccccaggtgg

SEQ ID NO: 54 - DNA, 23 bp, Artificial sequence
  a guide RNA (sgRNA) nucleic acid sequence
gtgcattattgcctgcctcctgg

SEQ ID NO: 55 - DNA, 23 bp, Artificial sequence
  a guide RNA (sgRNA) nucleic acid sequence
tacagccgcatctacttacgtgg

SEQ ID NO: 56 - DNA, 23 bp, Artificial sequence
  a guide RNA (sgRNA) nucleic acid sequence
aactaaggccgcggtcaaattgg

SEQ ID NO: 57 - DNA, 23 bp, Artificial sequence
  a guide RNA (sgRNA) nucleic acid sequence
aggtccaagagtgctcaagaggg

SEQ ID NO: 58 - DNA, 23 bp, Artificial sequence
  a guide RNA (sgRNA) nucleic acid sequence
ccctagaggacgtggcggtgagg

SEQ ID NO: 59 - DNA, 23 bp, Artificial sequence
  a guide RNA (sgRNA) nucleic acid sequence
cctcaccgccacgtcctctaggg

SEQ ID NO: 60 - DNA, 23 bp, Artificial sequence
  a guide RNA (sgRNA) nucleic acid sequence
gagtgtccaagccaggaggcagg

SEQ ID NO: 61 - DNA, 23 bp, Artificial sequence
  a guide RNA (sgRNA) nucleic acid sequence
agcggagtgtccaagccaggagg

SEQ ID NO: 62 - DNA, 23 bp, Artificial sequence
  a guide RNA (sgRNA) nucleic acid sequence
tcctcaccgccacgtcctctagg

SEQ ID NO: 63 - DNA, 23 bp, Artificial sequence
  a guide RNA (sgRNA) nucleic acid sequence
aggctgatgaactaaggccgcgg

SEQ ID NO: 64 - DNA, 23 bp, Artificial sequence
  a guide RNA (sgRNA) nucleic acid sequence
gtctcgagtgttgatcagtaagg

SEQ ID NO: 65 - DNA, 23 bp, Artificial sequence
  a guide RNA (sgRNA) nucleic acid sequence
tacgatcaaagctacatcggcgg

SEQ ID NO: 66 - DNA, 23 bp, Artificial sequence
  a guide RNA (sgRNA) nucleic acid sequence
ccactgtcgaggagttctggagg

SEQ ID NO: 67 - DNA, 23 bp, Artificial sequence
  a guide RNA (sgRNA) nucleic acid sequence
gccctcaacgccaatttgcttgg

SEQ ID NO: 68 - DNA, 23 bp, Artificial sequence
  a guide RNA (sgRNA) nucleic acid sequence
atcctgtttgtgcgagtggaggg

SEQ ID NO: 69 - DNA, 23 bp, Artificial sequence
  a guide RNA (sgRNA) nucleic acid sequence
gcgtttcttggttcgctttaggg

SEQ ID NO: 70 - DNA, 23 bp, Artificial sequence
  a guide RNA (sgRNA) nucleic acid sequence
cctccagaactcctcgacagtgg

SEQ ID NO: 71 - DNA, 23 bp, Artificial sequence
  a guide RNA (sgRNA) nucleic acid sequence
gctaaaccagcaatcgtagatgg

SEQ ID NO: 72 - DNA, 23 bp, Artificial sequence
  a guide RNA (sgRNA) nucleic acid sequence
atcggagaacagtttgactgtgg

SEQ ID NO: 73 - DNA, 23 bp, Artificial sequence
  a guide RNA (sgRNA) nucleic acid sequence
gcttcacagatgttcctcctagg

SEQ ID NO: 74 - DNA, 23 bp, Artificial sequence
  a guide RNA (sgRNA) nucleic acid sequence
ttattgcctgcctcctggcttgg

SEQ ID NO: 75 - DNA, 23 bp, Artificial sequence
  a guide RNA (sgRNA) nucleic acid sequence
ggtatatgcatggacctaggagg

SEQ ID NO: 76 - DNA, 23 bp, Artificial sequence
  a guide RNA (sgRNA) nucleic acid sequence
caacagctgcttgctatgatcgg

SEQ ID NO: 77 - DNA, 23 bp, Artificial sequence
  a guide RNA (sgRNA) nucleic acid sequence
tcgttaaaaagtaggtagcgtgg

SEQ ID NO: 78 - DNA, 23 bp, Artificial sequence
  a guide RNA (sgRNA) nucleic acid sequence
tctccactgtcgaggagttctgg

SEQ ID NO: 79 - DNA, 23 bp, Artificial sequence
  a guide RNA (sgRNA) nucleic acid sequence
actgttttccaatgctcgcctgg

SEQ ID NO: 80 - DNA, 23 bp, Artificial sequence
  a guide RNA (sgRNA) nucleic acid sequence
accgtgttgtttcctcgttcagg

SEQ ID NO: 81 - DNA, 23 bp, Artificial sequence
  a guide RNA (sgRNA) nucleic acid sequence
tgcttccaatgaagctgcgcagg

SEQ ID NO: 82 - DNA, 23 bp, Artificial sequence
  a guide RNA (sgRNA) nucleic acid sequence
cgatcatagcaagcagctgttgg

SEQ ID NO: 83 - DNA, 23 bp, Artificial sequence
  a guide RNA (sgRNA) nucleic acid sequence
acagtacttcattctggcagtgg

SEQ ID NO: 84 - DNA, 23 bp, Artificial sequence
  a guide RNA (sgRNA) nucleic acid sequence
acaagcggagtgtccaagccagg

SEQ ID NO: 85 - DNA, 23 bp, Artificial sequence
  a guide RNA (sgRNA) nucleic acid sequence
tctttgcatcatcctgaacgagg

SEQ ID NO: 86 - DNA, 23 bp, Artificial sequence
  a guide RNA (sgRNA) nucleic acid sequence
aaaagtaggtagcgtggaagtgg

SEQ ID NO: 87 - DNA, 23 bp, Artificial sequence
  a guide RNA (sgRNA) nucleic acid sequence
cttggtatatgcatggacctagg

SEQ ID NO: 88 - DNA, 23 bp, Artificial sequence
  a guide RNA (sgRNA) nucleic acid sequence
ttttttggccaggcgagcattgg

SEQ ID NO: 89 - DNA, 23 bp, Artificial sequence
  a guide RNA (sgRNA) nucleic acid sequence
tccaagcaaattggcgttgaggg

SEQ ID NO: 90 - DNA, 23 bp, Artificial sequence
  a guide RNA (sgRNA) nucleic acid sequence
taaggccgcggtcaaattggagg

SEQ ID NO: 91 - DNA, 23 bp, Artificial sequence
  a guide RNA (sgRNA) nucleic acid sequence
ggggaaaatctgataatggctgg

SEQ ID NO: 92 - DNA, 23 bp, Artificial sequence
  a guide RNA (sgRNA) nucleic acid sequence
aaggtccaagagtgctcaagagg

SEQ ID NO: 93 - DNA, 23 bp, Artificial sequence
  a guide RNA (sgRNA) nucleic acid sequence
gaagatcctgtttgtgcgagtgg

SEQ ID NO: 94 - DNA, 23 bp, Artificial sequence
  a guide RNA (sgRNA) nucleic acid sequence
ttccctccactcgcacaaacagg

SEQ ID NO: 95 - DNA, 23 bp, Artificial sequence
  a guide RNA (sgRNA) nucleic acid sequence
gggattatgaatgtgagggctgg

SEQ ID NO: 96 - DNA, 23 bp, Artificial sequence
  a guide RNA (sgRNA) nucleic acid sequence
tcactgatttctagagcttttgg

SEQ ID NO: 97 - DNA, 23 bp, Artificial sequence
  a guide RNA (sgRNA) nucleic acid sequence
ttcttgtcgtgggtcttgattgg

SEQ ID NO: 98 - DNA, 23 bp, Artificial sequence
  a guide RNA (sgRNA) nucleic acid sequence
atccaagcaaattggcgttgagg

SEQ ID NO: 99 - DNA, 23 bp, Artificial sequence
  a guide RNA (sgRNA) nucleic acid sequence
tgactcctccaatttgaccgcgg

SEQ ID NO: 100 - DNA, 23 bp, Artificial sequence
  a guide RNA (sgRNA) nucleic acid sequence
caagagtgctcaagagggtgagg

SEQ ID NO: 101 - DNA, 23 bp, Artificial sequence
  a guide RNA (sgRNA) nucleic acid sequence
gatcctgtttgtgcgagtggagg

SEQ ID NO: 102 - DNA, 23 bp, Artificial sequence
  a guide RNA (sgRNA) nucleic acid sequence
tgttaatgttagatctgggcagg

SEQ ID NO: 103 - DNA, 23 bp, Artificial sequence
  a guide RNA (sgRNA) nucleic acid sequence
ctataccttctccactgtcgagg

SEQ ID NO: 104 - DNA, 23 bp, Artificial sequence
  a guide RNA (sgRNA) nucleic acid sequence
gtgttgatcagtaaggaatgagg

SEQ ID NO: 105 - DNA, 23 bp, Artificial sequence
  a guide RNA (sgRNA) nucleic acid sequence
atcagtacagtacttcattctgg

SEQ ID NO: 106 - DNA, 23 bp, Artificial sequence
  a guide RNA (sgRNA) nucleic acid sequence
cttgaggaaggtgagatcgtcgg

SEQ ID NO: 107 - DNA, 23 bp, Artificial sequence
  a guide RNA (sgRNA) nucleic acid sequence
ctcaccctcttgagcactcttgg

SEQ ID NO: 108 - DNA, 23 bp, Artificial sequence
  a guide RNA (sgRNA) nucleic acid sequence
tgatgtggagtacattatgctgg

SEQ ID NO: 109 - DNA, 23 bp, Artificial sequence
  a guide RNA (sgRNA) nucleic acid sequence
cggtcaaattggaggagtcaagg

SEQ ID NO: 110 - DNA, 23 bp, Artificial sequence
  a guide RNA (sgRNA) nucleic acid sequence
caaatccaagcaagccacctggg

SEQ ID NO: 111 - DNA, 23 bp, Artificial sequence
  a guide RNA (sgRNA) nucleic acid sequence
gaactcctcgacagtggagaagg

SEQ ID NO: 112 - DNA, 23 bp, Artificial sequence
  a guide RNA (sgRNA) nucleic acid sequence
ttgagcactcttggaccttttgg

SEQ ID NO: 113 - DNA, 23 bp, Artificial sequence
  a guide RNA (sgRNA) nucleic acid sequence
tgatcgtcaagttttagcaacgg

SEQ ID NO: 114 - DNA, 23 bp, Artificial sequence
  a guide RNA (sgRNA) nucleic acid sequence
aatgaagtactgtactgatgtgg

SEQ ID NO: 115 - DNA, 23 bp, Artificial sequence
  a guide RNA (sgRNA) nucleic acid sequence
aatcaaaaccctagaggacgtgg

SEQ ID NO: 116 - DNA, 23 bp, Artificial sequence
  a guide RNA (sgRNA) nucleic acid sequence
tcctgaacgaggaaacaacacgg

SEQ ID NO: 117 - DNA, 23 bp, Artificial sequence
  a guide RNA (sgRNA) nucleic acid sequence
ggcgtttcttggttcgctttagg

SEQ ID NO: 118 - DNA, 23 bp, Artificial sequence
  a guide RNA (sgRNA) nucleic acid sequence
gcagttgttaatgttagatctgg

SEQ ID NO: 119 - DNA, 23 bp, Artificial sequence
  a guide RNA (sgRNA) nucleic acid sequence
gtattttgaaaaccctccttagg

SEQ ID NO: 120 - DNA, 23 bp, Artificial sequence
  a guide RNA (sgRNA) nucleic acid sequence
gtttttccatctacgattgctgg

SEQ ID NO: 121 - DNA, 23 bp, Artificial sequence
  a guide RNA (sgRNA) nucleic acid sequence
tagtgggaatacacaagcaaagg

SEQ ID NO: 122 - DNA, 23 bp, Artificial sequence
  a guide RNA (sgRNA) nucleic acid sequence
ccaaatccaagcaagccacctgg

SEQ ID NO: 123 - DNA, 23 bp, Artificial sequence
  a guide RNA (sgRNA) nucleic acid sequence
aggcgagcattggaaaacagtgg

SEQ ID NO: 124 - DNA, 23 bp, Artificial sequence
  a guide RNA (sgRNA) nucleic acid sequence
agtggactgtgaacttttcaagg

SEQ ID NO: 125 - DNA, 23 bp, Artificial sequence
  a guide RNA (sgRNA) nucleic acid sequence
cttgcttggatttggcagatggg

SEQ ID NO: 126 - DNA, 23 bp, Artificial sequence
  a guide RNA (sgRNA) nucleic acid sequence
tttgtgcgagtggagggaagtgg

SEQ ID NO: 127 - DNA, 23 bp, Artificial sequence
  a guide RNA (sgRNA) nucleic acid sequence
gctacaaaattaatggtacttgg

SEQ ID NO: 128 - DNA, 23 bp, Artificial sequence
  a guide RNA (sgRNA) nucleic acid sequence
atattccacgtaagtagatgcgg

SEQ ID NO: 129 - DNA, 23 bp, Artificial sequence
  a guide RNA (sgRNA) nucleic acid sequence
caaaaccctagaggacgtggcgg

SEQ ID NO: 130 - DNA, 23 bp, Artificial sequence
  a guide RNA (sgRNA) nucleic acid sequence
agggtgaggctgatgaactaagg

SEQ ID NO: 131 - DNA, 23 bp, Artificial sequence
  a guide RNA (sgRNA) nucleic acid sequence
gaatgtgttacggttctcaaagg

SEQ ID NO: 132 - DNA, 23 bp, Artificial sequence
  a guide RNA (sgRNA) nucleic acid sequence
acttggtgaggtagaatattagg

SEQ ID NO: 133 - DNA, 23 bp, Artificial sequence
  a guide RNA (sgRNA) nucleic acid sequence
tattacctgcgcagcttcattgg

SEQ ID NO: 134 - DNA, 23 bp, Artificial sequence
  a guide RNA (sgRNA) nucleic acid sequence
cagttgttaatgttagatctggg

SEQ ID NO: 135 - DNA, 23 bp, Artificial sequence
  a guide RNA (sgRNA) nucleic acid sequence
atgttagcagaatccatgctagg

SEQ ID NO: 136 - DNA, 23 bp, Artificial sequence
  a guide RNA (sgRNA) nucleic acid sequence
acagtggagaaggtatagatcgg

SEQ ID NO: 137 - DNA, 23 bp, Artificial sequence
  a guide RNA (sgRNA) nucleic acid sequence
ccaggtggcttgcttggatttgg

SEQ ID NO: 138 - DNA, 23 bp, Artificial sequence
  a guide RNA (sgRNA) nucleic acid sequence
ttgttgtaaacacttgaagcagg

SEQ ID NO: 139 - DNA, 23 bp, Artificial sequence
  a guide RNA (sgRNA) nucleic acid sequence
tgaatgtgagggctggagagagg

SEQ ID NO: 140 - DNA, 23 bp, Artificial sequence
  a guide RNA (sgRNA) nucleic acid sequence
aaatccaagcaagccacctgggg

SEQ ID NO: 141 - DNA, 23 bp, Artificial sequence
  a guide RNA (sgRNA) nucleic acid sequence
caaattgaacatggaattcgggg

SEQ ID NO: 142 - DNA, 23 bp, Artificial sequence
  a guide RNA (sgRNA) nucleic acid sequence
agatacgatcaaagctacatcgg

SEQ ID NO: 143 - DNA, 23 bp, Artificial sequence
  a guide RNA (sgRNA) nucleic acid sequence
aattaagggtgaatgtgttacgg

SEQ ID NO: 144 - DNA, 23 bp, Artificial sequence
  a guide RNA (sgRNA) nucleic acid sequence
tgttgtaaacacttgaagcaggg

SEQ ID NO: 145 - DNA, 23 bp, Artificial sequence
  a guide RNA (sgRNA) nucleic acid sequence
tcaaggggaaaatctgataatgg

SEQ ID NO: 146 - DNA, 23 bp, Artificial sequence
  a guide RNA (sgRNA) nucleic acid sequence
tcctaaacaaaaccctaaggagg

SEQ ID NO: 147 - DNA, 23 bp, Artificial sequence
  a guide RNA (sgRNA) nucleic acid sequence
ggctattgttgctacctacctgg

SEQ ID NO: 148 - DNA, 23 bp, Artificial sequence
  a guide RNA (sgRNA) nucleic acid sequence
gagagcagctacaaaattaatgg

SEQ ID NO: 149 - DNA, 23 bp, Artificial sequence
  a guide RNA (sgRNA) nucleic acid sequence
tattttgaaaaccctccttaggg

SEQ ID NO: 150 - DNA, 23 bp, Artificial sequence
  a guide RNA (sgRNA) nucleic acid sequence
ctctacattccttgtgtgattgg

SEQ ID NO: 151 - DNA, 23 bp, Artificial sequence
  a guide RNA (sgRNA) nucleic acid sequence
gcttgcttggatttggcagatgg

SEQ ID NO: 152 - DNA, 23 bp, Artificial sequence
  a guide RNA (sgRNA) nucleic acid sequence
tctagggttttgattagcaatgg

SEQ ID NO: 153 - DNA, 23 bp, Artificial sequence
  a guide RNA (sgRNA) nucleic acid sequence
agctcctaaacaaaaccctaagg

SEQ ID NO: 154 - DNA, 23 bp, Artificial sequence
  a guide RNA (sgRNA) nucleic acid sequence
ttatgctggagaaaatatctggg

SEQ ID NO: 155 - DNA, 23 bp, Artificial sequence
  a guide RNA (sgRNA) nucleic acid sequence
ggcaggataaaatatcgatttgg

SEQ ID NO: 156 - DNA, 23 bp, Artificial sequence
  a guide RNA (sgRNA) nucleic acid sequence
cttgattacaatgagagcattgg

SEQ ID NO: 157 - DNA, 23 bp, Artificial sequence
  a guide RNA (sgRNA) nucleic acid sequence
tgtcgtgggtcttgattggatgg

SEQ ID NO: 158 - DNA, 23 bp, Artificial sequence
  a guide RNA (sgRNA) nucleic acid sequence
tggactgtgaacttttcaagggg

SEQ ID NO: 159 - DNA, 23 bp, Artificial sequence
  a guide RNA (sgRNA) nucleic acid sequence
gagcattggaaaacagtggaagg

SEQ ID NO: 160 - DNA, 23 bp, Artificial sequence
  a guide RNA (sgRNA) nucleic acid sequence
aatggtcttaatctctctcaagg

SEQ ID NO: 161 - DNA, 23 bp, Artificial sequence
  a guide RNA (sgRNA) nucleic acid sequence
tgatattcttggtatatgcatgg

SEQ ID NO: 162 - DNA, 23 bp, Artificial sequence
  a guide RNA (sgRNA) nucleic acid sequence
ttttcttttcaaacctagcatgg

SEQ ID NO: 163 - DNA, 23 bp, Artificial sequence
  a guide RNA (sgRNA) nucleic acid sequence
aaggaaatgagttttagctatgg

SEQ ID NO: 164 - DNA, 23 bp, Artificial sequence
  a guide RNA (sgRNA) nucleic acid sequence
gttggtctagtggtcaattaagg

SEQ ID NO: 165 - DNA, 23 bp, Artificial sequence
  a guide RNA (sgRNA) nucleic acid sequence
gaatttgtagcttgattccaagg

SEQ ID NO: 166 - DNA, 23 bp, Artificial sequence
  a guide RNA (sgRNA) nucleic acid sequence
acaaaggatcaagctgaaattgg

SEQ ID NO: 167 - DNA, 23 bp, Artificial sequence
  a guide RNA (sgRNA) nucleic acid sequence
attatgctggagaaaatatctgg

SEQ ID NO: 168 - DNA, 23 bp, Artificial sequence
  a guide RNA (sgRNA) nucleic acid sequence
tcaaattgaacatggaattcggg

SEQ ID NO: 169 - DNA, 23 bp, Artificial sequence
  a guide RNA (sgRNA) nucleic acid sequence
ccctccttagggttttgtttagg

SEQ ID NO: 170 - DNA, 23 bp, Artificial sequence
  a guide RNA (sgRNA) nucleic acid sequence
gtggactgtgaacttttcaaggg

SEQ ID NO: 171 - DNA, 23 bp, Artificial sequence
  a guide RNA (sgRNA) nucleic acid sequence
tattcatcatccaagcaaattgg

SEQ ID NO: 172 - DNA, 23 bp, Artificial sequence
  a guide RNA (sgRNA) nucleic acid sequence
ttggtctagtggtcaattaaggg

SEQ ID NO: 173 - DNA, 23 bp, Artificial sequence
  a guide RNA (sgRNA) nucleic acid sequence
caaacaggatcttcccatttagg

SEQ ID NO: 174 - DNA, 23 bp, Artificial sequence
  a guide RNA (sgRNA) nucleic acid sequence
cctaaacaaaaccctaaggaggg

SEQ ID NO: 175 - DNA, 23 bp, Artificial sequence
  a guide RNA (sgRNA) nucleic acid sequence
tgctaacatcgttaaaaagtagg

SEQ ID NO: 176 - DNA, 23 bp, Artificial sequence
  a guide RNA (sgRNA) nucleic acid sequence
acataaaattgagcctaaatggg

SEQ ID NO: 177 - DNA, 23 bp, Artificial sequence
  a guide RNA (sgRNA) nucleic acid sequence
attgctaatcaaaaccctagagg

SEQ ID NO: 178 - DNA, 23 bp, Artificial sequence
  a guide RNA (sgRNA) nucleic acid sequence
tcaaatggatatcaaacaagcgg

SEQ ID NO: 179 - DNA, 23 bp, Artificial sequence
  a guide RNA (sgRNA) nucleic acid sequence
cgttctatttttttttggccagg

SEQ ID NO: 180 - DNA, 23 bp, Artificial sequence
  a guide RNA (sgRNA) nucleic acid sequence
tcaaaatattaaaaccaggtagg

SEQ ID NO: 181 - DNA, 23 bp, Artificial sequence
  a guide RNA (sgRNA) nucleic acid sequence
gttgtatttgattggaagtaagg

SEQ ID NO: 182 - DNA, 23 bp, Artificial sequence
  a guide RNA (sgRNA) nucleic acid sequence
gaaatgagttttagctatggtgg

SEQ ID NO: 183 - DNA, 23 bp, Artificial sequence
  a guide RNA (sgRNA) nucleic acid sequence
acaacacttcttcttttgcttgg

SEQ ID NO: 184 - DNA, 23 bp, Artificial sequence
  a guide RNA (sgRNA) nucleic acid sequence
gaatgtgagggctggagagaggg

SEQ ID NO: 185 - DNA, 23 bp, Artificial sequence
  a guide RNA (sgRNA) nucleic acid sequence
agcagaataagcatcaaaacagg

SEQ ID NO: 186 - DNA, 23 bp, Artificial sequence
  a guide RNA (sgRNA) nucleic acid sequence
gttcgctttagggaaaatttagg

SEQ ID NO: 187 - DNA, 23 bp, Artificial sequence
  a guide RNA (sgRNA) nucleic acid sequence
tagggaaaatttaggaaacttgg

SEQ ID NO: 188 - DNA, 23 bp, Artificial sequence
  a guide RNA (sgRNA) nucleic acid sequence
ctaatgaaggcgtaaaaaaatgg

SEQ ID NO: 189 - DNA, 23 bp, Artificial sequence
  a guide RNA (sgRNA) nucleic acid sequence
gaaaagaagaacaatggtcgagg

SEQ ID NO: 190 - DNA, 23 bp, Artificial sequence
  a guide RNA (sgRNA) nucleic acid sequence
aagaagggattatgaatgtgagg

SEQ ID NO: 191 - DNA, 23 bp, Artificial sequence
  a guide RNA (sgRNA) nucleic acid sequence
gtaaggaatgaggatgattgagg

SEQ ID NO: 192 - DNA, 23 bp, Artificial sequence
  a guide RNA (sgRNA) nucleic acid sequence
acatgcgaagaataaatatatgg

SEQ ID NO: 193 - DNA, 23 bp, Artificial sequence
  a guide RNA (sgRNA) nucleic acid sequence
attgatcaaccaatcacacaagg

SEQ ID NO: 194 - DNA, 23 bp, Artificial sequence
  a guide RNA (sgRNA) nucleic acid sequence
gtcaaattgaacatggaattcgg

SEQ ID NO: 195 - DNA, 23 bp, Artificial sequence
  a guide RNA (sgRNA) nucleic acid sequence
cttatgtgtcaaattgaacatgg

SEQ ID NO: 196 - DNA, 23 bp, Artificial sequence
  a guide RNA (sgRNA) nucleic acid sequence
gactgtggtgatgaaatttgtgg

SEQ ID NO: 197 - DNA, 23 bp, Artificial sequence
  a guide RNA (sgRNA) nucleic acid sequence
agatgggttatcaaaccaaaagg

SEQ ID NO: 198 - DNA, 23 bp, Artificial sequence
  a guide RNA (sgRNA) nucleic acid sequence
aaaatttaggaaacttggtgagg

SEQ ID NO: 199 - DNA, 23 bp, Artificial sequence
  a guide RNA (sgRNA) nucleic acid sequence
agaagggattatgaatgtgaggg

SEQ ID NO: 200 - DNA, 23 bp, Artificial sequence
  a guide RNA (sgRNA) nucleic acid sequence
tatattatgagttggtctagtgg

SEQ ID NO: 201 - DNA, 23 bp, Artificial sequence
  a guide RNA (sgRNA) nucleic acid sequence
acgtggcggtgaggaagatgagg

SEQ ID NO: 202 - DNA, 23 bp, Artificial sequence
  a guide RNA (sgRNA) nucleic acid sequence
aatgtgagggctggagagagggg

SEQ ID NO: 203 - DNA, 23 bp, Artificial sequence
  a guide RNA (sgRNA) nucleic acid sequence
tgtataatgtagaaacttgaagg

SEQ ID NO: 204 - DNA, 23 bp, Artificial sequence
  a guide RNA (sgRNA) nucleic acid sequence
ttgttttttttggcgtttcttgg

SEQ ID NO: 205 - DNA, 23 bp, Artificial sequence
  a guide RNA (sgRNA) nucleic acid sequence
tgtacaattttaactaatgaagg

SEQ ID NO: 206 - DNA, 23 bp, Artificial sequence
  a guide RNA (sgRNA) nucleic acid sequence
aacataaaattgagcctaaatgg

SEQ ID NO: 207 - DNA, 23 bp, Artificial sequence
  a guide RNA (sgRNA) nucleic acid sequence
ttcttttcagcaactaaatttgg

SEQ ID NO: 208 - DNA, 23 bp, Artificial sequence
  a guide RNA (sgRNA) nucleic acid sequence
aaaagctttcatgaagatgacgg

SEQ ID NO: 209 - DNA, 23 bp, Artificial sequence
  a guide RNA (sgRNA) nucleic acid sequence
tcattagttaaaattgtacatgg

SEQ ID NO: 210 - DNA, 23 bp, Artificial sequence
  a guide RNA (sgRNA) nucleic acid sequence
gaatttttattttcttgtcgtgg

SEQ ID NO: 211 - DNA, 23 bp, Artificial sequence
  a guide RNA (sgRNA) nucleic acid sequence
caactaaatttggatgagtttgg

SEQ ID NO: 212 - DNA, 23 bp, Artificial sequence
  a guide RNA (sgRNA) nucleic acid sequence
gtattagaaacatgaaacaaagg

SEQ ID NO: 213 - DNA, 23 bp, Artificial sequence
  a guide RNA (sgRNA) nucleic acid sequence
gaagtaaggtaagacaaagaagg

SEQ ID NO: 214 - DNA, 23 bp, Artificial sequence
  a guide RNA (sgRNA) nucleic acid sequence
actgtcaaaatattaaaaccagg

SEQ ID NO: 215 - DNA, 23 bp, Artificial sequence
  a guide RNA (sgRNA) nucleic acid sequence
aaccatttgatttctatttctgg

SEQ ID NO: 216 - DNA, 23 bp, Artificial sequence
  a guide RNA (sgRNA) nucleic acid sequence
gatccttatttgtttttggatgg

SEQ ID NO: 217 - DNA, 23 bp, Artificial sequence
  a guide RNA (sgRNA) nucleic acid sequence
gaccagaaatagaaatcaaatgg

SEQ ID NO: 218 - DNA, 23 bp, Artificial sequence
  a guide RNA (sgRNA) nucleic acid sequence
ttactcttgttgtatttgattgg

SEQ ID NO: 219 - DNA, 23 bp, Artificial sequence
  a guide RNA (sgRNA) nucleic acid sequence
aagtaaggtaagacaaagaaggg

SEQ ID NO: 220 - DNA, 23 bp, Artificial sequence
  a guide RNA (sgRNA) nucleic acid sequence
aaataaattacttgatcttgtgg

SEQ ID NO: 221 - DNA, 23 bp, Artificial sequence
  a guide RNA (sgRNA) nucleic acid sequence
ttttggatggtttggttttaggg

SEQ ID NO: 222 - DNA, 23 bp, Artificial sequence
  a guide RNA (sgRNA) nucleic acid sequence
gttgctgaaaagaagaacaatgg

SEQ ID NO: 223 - DNA, 23 bp, Artificial sequence
  a guide RNA (sgRNA) nucleic acid sequence
acaaaacagagcaaataagaagg

SEQ ID NO: 224 - DNA, 23 bp, Artificial sequence
  a guide RNA (sgRNA) nucleic acid sequence
gaattaaatatattatgagttgg

SEQ ID NO: 225 - DNA, 23 bp, Artificial sequence
  a guide RNA (sgRNA) nucleic acid sequence
ttaagatccttatttgtttttgg

SEQ ID NO: 226 - DNA, 23 bp, Artificial sequence
  a guide RNA (sgRNA) nucleic acid sequence
taacacgttctatttttttttgg

SEQ ID NO: 227 - DNA, 23 bp, Artificial sequence
  a guide RNA (sgRNA) nucleic acid sequence
aatttttattttcttgtcgtggg

SEQ ID NO: 228 - DNA, 23 bp, Artificial sequence
  a guide RNA (sgRNA) nucleic acid sequence
aagaaggaaggaggcaaagatgg

SEQ ID NO: 229 - DNA, 23 bp, Artificial sequence
  a guide RNA (sgRNA) nucleic acid sequence
ttatttgtttttggatggtttgg

SEQ ID NO: 230 - DNA, 23 bp, Artificial sequence
  a guide RNA (sgRNA) nucleic acid sequence
aacagagcaaataagaaggaagg

SEQ ID NO: 231 - DNA, 23 bp, Artificial sequence
  a guide RNA (sgRNA) nucleic acid sequence
agagcaaataagaaggaaggagg

SEQ ID NO: 232 - DNA, 23 bp, Artificial sequence
  a guide RNA (sgRNA) nucleic acid sequence
ttatttgctctgttttgttgtgg

SEQ ID NO: 233 - DNA, 23 bp, Artificial sequence
  a guide RNA (sgRNA) nucleic acid sequence
tttttggatggtttggttttagg

SEQ ID NO: 234 - DNA, 23 bp, Artificial sequence
  a guide RNA (sgRNA) nucleic acid sequence
aaaccatccaaaaacaaataagg

SEQ ID NO: 235 - DNA, 23 bp, Artificial sequence
  a guide RNA (sgRNA) nucleic acid sequence
attcaaaaaaataatctatatgg

SEQ ID NO: 236 - DNA, 23 bp, Artificial sequence
  a guide RNA (sgRNA) nucleic acid sequence
aatcaaatggttatcagacaggg

SEQ ID NO: 237 - DNA, 23 bp, Artificial sequence
  a guide RNA (sgRNA) nucleic acid sequence
ttttttttttttaagaaccttgg

SEQ ID NO: 238 - DNA, 23 bp, Artificial sequence
  a guide RNA (sgRNA) nucleic acid sequence
gatggaaaaacaagaaataaggg

SEQ ID NO: 239 - DNA, 23 bp, Artificial sequence
  a guide RNA (sgRNA) nucleic acid sequence
ttctaacaatttctttataatgg

SEQ ID NO: 240 - DNA, 23 bp, Artificial sequence
  a guide RNA (sgRNA) nucleic acid sequence
tgataatggctggttgtacacgg

SEQ ID NO: 241 - DNA, 23 bp, Artificial sequence
  a guide RNA (sgRNA) nucleic acid sequence
gaagatgaggaacttgaggaagg

SEQ ID NO: 242 - DNA, 23 bp, Artificial sequence
  a guide RNA (sgRNA) nucleic acid sequence
tcattatatgcttaatttttagg

SEQ ID NO: 243 - DNA, 23 bp, Artificial sequence
  a guide RNA (sgRNA) nucleic acid sequence
tgaggaagatgaggaacttgagg

SEQ ID NO: 244 - DNA, 23 bp, Artificial sequence
  a guide RNA (sgRNA) nucleic acid sequence
attttttcttttgatattcttgg

SEQ ID NO: 245 - DNA, 23 bp, Artificial sequence
  a guide RNA (sgRNA) nucleic acid sequence
agatggaaaaacaagaaataagg

SEQ ID NO: 246 - DNA, 23 bp, Artificial sequence
  a guide RNA (sgRNA) nucleic acid sequence
atttttcttaaaattttagtggg

SEQ ID NO: 247 - DNA, 23 bp, Artificial sequence
  a guide RNA (sgRNA) nucleic acid sequence
aaacaagaaataagggaaaacgg

SEQ ID NO: 248 - DNA, 23 bp, Artificial sequence
  a guide RNA (sgRNA) nucleic acid sequence
aaatcaaatggttatcagacagg

SEQ ID NO: 249 - DNA, 23 bp, Artificial sequence
  a guide RNA (sgRNA) nucleic acid sequence
tatttttcttaaaattttagtgg

SEQ ID NO: 250 - DNA, 23 bp, Artificial sequence
  a guide RNA (sgRNA) nucleic acid sequence
ttcaaaatacatacaaaaaaagg

SEQ ID NO: 251 - DNA, 23 bp, Artificial sequence
  a guide RNA (sgRNA) nucleic acid sequence
agttaaaaataagataaaatagg

SEQ ID NO: 252 - DNA, 23 bp, Artificial sequence
  a guide RNA (sgRNA) nucleic acid sequence
atctaaaaccaaaaatcaaatgg

SEQ ID NO: 253 - DNA, 23 bp, Artificial sequence
  a guide RNA (sgRNA) nucleic acid sequence
tcgattctttttgttttttttgg

SEQ ID NO: 254 - DNA, 15 bp, Artificial sequence
  a guide RNA (sgRNA) nucleic acid sequence
tttgatatccatttg

SEQ ID NO: 255 - DNA, 23 bp, Artificial sequence
  a guide RNA (sgRNA) nucleic acid sequence
gaaaaccctagaggacgtggcgg

SEQ ID NO: 256 - DNA, 24 bp, Artificial sequence
  a guide RNA (sgRNA) nucleic acid sequence
gcagttgttaatgttagatctggg

SEQ ID NO: 257 - DNA, 72 bp, Artificial sequence
  sequencing product
ctttctaattccattgctaatcaaaaccctagaggacgtggcggtgaggaagatgaggaacttgaggaagwt

SEQ ID NO: 258 - DNA, 50 bp, Artificial sequence
  sequencing product
ctttctaattccattgctaatcaaaaccctagatgaggaacttgaggaag

SEQ ID NO: 259 - DNA, 50 bp, Artificial sequence
  sequencing product
ctttttaattccattgctaatcaaaaccctagatgaggagcttgaggaag

SEQ ID NO: 260 - DNA, 69 bp, Artificial sequence
  sequencing product
ctttctaattccattgctaatcaaaaccctagaggactggcggtgaggaagatgaggaacttgaggaag

SEQ ID NO: 261 - DNA, 68 bp, Artificial sequence
  sequencing product
ctaattccattgctaatcaaaaccctagaggacgtggcggtgaggaagatgaggaacttgaggaagwt

SEQ ID NO: 262 - DNA, 46 bp, Artificial sequence
  sequencing product
ctaattccattgctaatcaaaaccctagatgaggaacttgaggaag

SEQ ID NO: 263 - DNA, 68 bp, Artificial sequence; identical to SEQ ID NO: 261
  sequencing product

SEQ ID NO: 264 - DNA, 65 bp, Artificial sequence
  sequencing product
ctaattccattgctaatcaaaaccctagaggactggcggtgaggaagatgaggaacttgaggaag

SEQ ID NO: 265 - DNA, 68 bp, Artificial sequence; identical to SEQ ID NO: 261
  sequencing product

SEQ ID NO: 266 - DNA, 65 bp, Artificial sequence; identical to SEQ ID NO: 264
  sequencing product

SEQ ID NO: 267 - DNA, 68 bp, Artificial sequence; identical to SEQ ID NO: 261
  sequencing product

SEQ ID NO: 268 - DNA, 46 bp, Artificial sequence; identical to SEQ ID NO: 262
  sequencing product

SEQ ID NO: 269 - DNA, 65 bp, Artificial sequence; identical to SEQ ID NO: 264
  sequencing product

SEQ ID NO: 270 - DNA, 58 bp, Artificial sequence
  sequencing product
ctaattccattgctaatcaaaaccctagaggacgtggcggtgaggaagatgaggaawt

SEQ ID NO: 271 - DNA, 41 bp, Artificial sequence
  sequencing product
ctaattccattgctaatcatggcggtgaggaagatgaggaa

SEQ ID NO: 272 - DNA, 47 bp, Artificial sequence
  sequencing product
ctaattccattgctaatcaaaaccctggcggtgaggaagatgaggaa

SEQ ID NO: 273 - DNA, 51 bp, Artificial sequence
  sequencing product
ctaattccattgctaatcaaaaccctagagggcggtgaggaagatgaggaa

SEQ ID NO: 274 - DNA, 45 bp, Artificial sequence
  sequencing product
ctaattccattgctaatcaaaactggcggtgaggaagatgaggaa

SEQ ID NO: 275 - DNA, 47 bp, Artificial sequence
  sequencing product
ctaattccattgctaatcaaaaccgtggcggtgaggaagatgaggaa

SEQ ID NO: 276 - DNA, 58 bp, Artificial sequence; identical to SEQ ID NO: 270
  sequencing product

SEQ ID NO: 277 - DNA, 55 bp, Artificial sequence
  sequencing product
ctaattccattgctaatcaaaaccctagaggatgggcggtgaggaagatgtggaa

SEQ ID NO: 278 - DNA, 55 bp, Artificial sequence
  sequencing product
ctaattccattgctaatcaaaaccctagaggacgggcggtgaggaagatgaggaa

SEQ ID NO: 279 - DNA, 55 bp, Artificial sequence
  sequencing product
ctaattccattgctaatcaaaaccctagaggactggcggtgaggaagatgaggaa

SEQ ID NO: 280 - DNA, 54 bp, Artificial sequence
  sequencing product
ctaattccattgctaatcaaaaccctagaggatggcggtgaggaagatgaggaa

SEQ ID NO: 281 - DNA, 56 bp, Artificial sequence
  sequencing product
ctaattccattgctaatcaaaaccctagaggacgtggcggtgaggaagatgaggaa

SEQ ID NO: 282 - DNA, 41 bp, Artificial sequence; identical to SEQ ID NO: 271
  sequencing product

SEQ ID NO: 283 - DNA, 47 bp, Artificial sequence; identical to SEQ ID NO: 272
  sequencing product

SEQ ID NO: 284 - DNA, 55 bp, Artificial sequence; identical to SEQ ID NO: 279
  sequencing product

SEQ ID NO: 285 - DNA, 55 bp, Artificial sequence
  sequencing product
ctaattccattgctaatcaaaaccctagagggcgggcggtgaggaagatgaggaa

SEQ ID NO: 286 - DNA, 51 bp, Artificial sequence
  sequencing product
ctaattccattgctgatcaaaaccctagaggacggtgaggaagatgaggaa

SEQ ID NO: 287 - DNA, 53 bp, Artificial sequence
  sequencing product
ctaattccattgctaatcaaaaccctagaggtggcggtgaggaagatgaggaa

SEQ ID NO: 288 - DNA, 54 bp, Artificial sequence; identical to SEQ ID NO: 280
  sequencing product

SEQ ID NO: 289 - DNA, 69 bp, Artificial sequence
  sequencing product
gtggtgatgaaatttgtggagcagttgttaatgttagatctgggcaggataaaatatcgatttggacwt

SEQ ID NO: 290 - DNA, 65 bp, Artificial sequence
  sequencing product
gtggtgatgaaatttgtggagcagttgttaatgttagatgggcaggataaaatatcgatttggac

SEQ ID NO: 291 - DNA, 68 bp, Artificial sequence
  sequencing product
gtggtgatgaaatttgtggagcagttgttaatgttagagtctgggcaggataaaatatcgatttggac

SEQ ID NO: 292 - DNA, 69 bp, Artificial sequence; identical to SEQ ID NO: 289
  sequencing product

SEQ ID NO: 293 - DNA, 63 bp, Artificial sequence
  sequencing product
gtggtgatgaaatttgtggagcagttgttaatgttctgggcaggataaaatatcgatttggac

SEQ ID NO: 294 - DNA, 66 bp, Artificial sequence
  sequencing product
gtggtgatgaaatttgtggagcagttgttaatgttagtctgggcaggataaaatatcgatttggac

SEQ ID NO: 295 - DNA, 69 bp, Artificial sequence; identical to SEQ ID NO: 289
  sequencing product

SEQ ID NO: 296 - DNA, 64 bp, Artificial sequence
  sequencing product
gtggtgatgaaatttgtggagcagttgttaatgtttctgggcaggataaaatatcgatttggac

SEQ ID NO: 297 - DNA, 62 bp, Artificial sequence
  sequencing product
gtggtgatgaaatttgtggagcagttgttaatgtctgggcaggataaaatatcgatttggac

SEQ ID NO: 298 - DNA, 69 bp, Artificial sequence; identical to SEQ ID NO: 289
  sequencing product

SEQ ID NO: 299 - DNA, 65 bp, Artificial sequence
  sequencing product
gtggtgatgaaatttgtggagcagttgttaatgttatctgggcaggataaaatatcgatttggac

SEQ ID NO: 300 - DNA, 63 bp, Artificial sequence
  sequencing product
gtggtgatgaaatttgtggagcagttgttaatgttatgggcaggataaaatatcgatttggac